(19) United States
(12) Patent Application Publication
(10) Pub. No.: US 2006/0013875 A1
(43) Pub. Date: Jan. 19, 2006

(54) Combination immediate release controlled release levodopa/carbidopa dosage forms

(75) Inventors: Chien-Hsuan Han, Sunnyvale, CA (US); Ann F. Hsu, Los Altos Hills, CA (US); Larry Hsu, Los Altos, CA (US)

Correspondence Address: FITCH, EVEN, TABIN & FLANNERY;Suite 401L, 1801 K Street, NW, Washington, DC, 20006, US

(73) Assignee: IMPAX Laboratories, Inc.

(21) Appl. No.: 11/194,569

(22) Filed: Aug. 2, 2005

Related U.S. Application Data
(63) Continuation-in-part of application No. 10/241,837, filed on Sep. 12, 2002.
Application No. 10/241,837 is a continuation-in-part of application No. 10/158,412, filed on May 29, 2002, now abandoned.

(51) Int. Cl. A61K 9/22; A61K 31/198

(52) U.S. Cl. 424/468; 514/567

(57) ABSTRACT

The present invention relates to pharmaceutical dosage forms of a combination of carbidopa and levodopa comprising both immediate release and controlled release components for the treatment of ailments associated with depleted amounts of dopamine in a patient's brain tissue, which dosage forms display no food effect or at least substantially avoid the food effect, and a related method of treatment for a patient in which the bioavailability of levodopa under non-fasting conditions is equivalent to the bioavailability under fasting conditions.

### RELATED APPLICATIONS

This application is a continuation in part of U.S. patent application Ser. No. 10/241,837, filed Sep. 12, 2002, which is a continuation in part of U.S. patent application Ser. No. 10/158,412, filed on May 29, 2002, both by Chien-Hsuan Han et al., both entitled Combination Immediate Release Sustained Release Levodopa/Carbidopa Dosage Forms, the complete disclosures of which are incorporated herein by reference in their entireties, and claims the benefit of said applications under 35 U.S.C. §120.

### FIELD OF THE INVENTION

The present invention relates to dosage forms of a combination of carbidopa and levodopa comprising both immediate release and controlled release components for the treatment of ailments associated with depleted amounts of dopamine in a patient's brain tissue, and especially for treating a patient in need of such treatment, under fasting or non-fasting conditions.

### BACKGROUND

Combinations of carbidopa and levodopa to treat Parkinson's disease are known in the pharmaceutical arts. Several products currently on the North American market, including SINEMET® and SINEMET® CR, contain combinations of carbidopa and levodopa in immediate release and controlled release forms, respectively. Overseas, other decarboxylase inhibitor and levodopa combinations include those sold under the mark MADOPAR® (levodopa with benserazide instead of carbidopa).

The carbidopa and levodopa combination is used to treat the symptoms of Parkinson's disease, which is characterized by abnormally low levels of dopamine. Dopamine is a neurotransmitter having significant influence over the mobility and control of the skeletal muscular system. Patients suffering from Parkinson's disease frequently have periods in which their mobility becomes difficult, often resulting in an inability to move.

Administering dopamine is not effective to treat Parkinson's disease because dopamine does not cross the blood brain barrier. To resolve this failure, Parkinson's patients are administered levodopa, the metabolic precursor of dopamine. Levodopa crosses the blood brain barrier and is rapidly converted to dopamine, thereby alleviating the symptoms of Parkinson's disease caused by reduced levels of dopamine. Levodopa is problematic because of its rapid decarboxylation by tissues other than the brain. Thus, when levodopa is administered alone, large doses are required because only a small portion is transported to the brain unchanged.

Patients treated with levodopa therapy for Parkinson's disease may frequently develop motor fluctuations characterized by end-of-dose failure, peak dose dyskinesia and akinesia. An advanced form of motor fluctuations is known as the “on-off effect” in which the patient suffers from unpredictable swings from mobility to immobility. It is believed that the on-off effect can be minimized in some patients with a treatment regimen which produces narrow ranges of plasma levels of levodopa.

Aromatic amino acid decarboxylase (AAAD) inhibitors, such as carbidopa, inhibit peripheral levodopa decarboxylation.

Pharmaceutical combinations of carbidopa and levodopa are available for the treatment of Parkinson's disease, including SINEMET® and SINEMET® CR, as well as products containing levodopa and an alternative AAAD inhibitor, such as benserazide, of which the MADOPAR® product is an example. Additionally, some levodopa/carbidopa formulations also include a catechol O-methyltransferase (COMT) inhibitor, such as entacapone, to block the function of another levodopa-degrading enzyme. When entacapone is given in conjunction with levodopa and an AAAD inhibitor, metabolism of levodopa is reduced and plasma levels of levodopa are greater and more sustained compared to administration of levodopa and an AAAD inhibitor alone. For example, when 200 mg of entacapone is administered together with a combination of immediate release carbidopa and levodopa, it increases levodopa blood plasma level, measured as the area under the curve (AUC), by about 35 percent, and prolongs the levodopa half-life from 1.3 hours to 2.4 hours. (COMTAN (entacapone) Package Insert, Orion Corporation, Orion Pharma (Espoo, Finland) and Novartis Pharmaceuticals Corporation (East Hanover, N.J.) (1999)). An example of a formulation of carbidopa, levodopa, and entacapone for the treatment of Parkinson's disease is STALEVO® (Novartis Pharmaceuticals USA). Other examples of COMT inhibitors are CGP-28014 and tolcapone.

Carbidopa inhibits the decarboxylation of levodopa by a patient's body tissues outside of the brain. Small doses of carbidopa administered in conjunction with levodopa allow a larger percentage of levodopa to reach the brain unchanged for later conversion to dopamine. There is at least one study reporting that carbidopa reduces the oral dose of levodopa required to produce a given response by about 75% and, when administered in conjunction with levodopa, increases plasma levels and the plasma half life of levodopa. (SINEMET® (carbidopa/levodopa) Package Insert, Merck & Co., Inc. and Bristol-Myers Squibb (2002)). The carbidopa and levodopa combination allows for lower doses of levodopa with a concordant reduction of side effects associated with dopamine generated by decarboxylation of levodopa peripherally.

The carbidopa and levodopa combination product is now available in immediate release as well as controlled release formulations. The controlled release formulations allow for the continuous release of drug over a prolonged period in an attempt to maintain tight plasma levodopa concentration ranges. However, the use of controlled release dosage forms is problematic in that its absorption is slow as compared to immediate release formulations. Many Parkinson's patients wake up in the morning having little or no mobility due to the wearing off of the previous dose taken the day or evening before. Once the previous dose has worn off, such patients are usually unwilling or unable to wait for the extended period of time required for a controlled release dosage form to deliver the appropriate plasma levels of levodopa. Although immediate release formulations offer immediate absorption of levodopa and rapid onset of effect, the use of immediate release formulations requires more frequent dosing and is associated with frequent fluctuating plasma levodopa concentrations.

Food can induce changes in a patient's gastrointestinal tract and also alter lumenal metabolism and even physically or chemically interact with a drug. It follows that the effects of food are complicated and difficult to predict and are influenced by many variables. It is undesirable if the bioavailability of a drug substance differs depending on whether a patient is in a fed or fasting state. These and other considerations are germane insofar as levodopa and carbidopa formulations are concerned because formulations currently on the market, e.g., SINEMET®, are prescribed with labeling that draws attention to the food effect and specifies dosing instructions against administration relative to meals. For example, the labeling for SINEMET® immediate release carbidopa/levodopa tablets suggests that consumption of high-protein foods may delay and decrease levodopa absorption, while the labeling for SINEMET® CR (controlled release tablets) states that the availability and peak concentrations of levodopa increase when administered with food. (SINEMET® (carbidopa/levodopa) Package Insert, Merck & Co., Inc. and Bristol-Myers Squibb (2002); SINEMET® CR (carbidopa/levodopa) Package Insert, Merck & Co., Inc. and Bristol-Myers Squibb Company (2002)).

Therefore, a continuing need remains unmet for carbidopa and levodopa products with both immediate release and controlled release properties which will improve the administration of levodopa to Parkinson's patients by reducing variation in bioavailability of levodopa on an intra-patient basis, regardless of whether the patient is in a fed or fasting state.

### SUMMARY OF THE INVENTION

Accordingly, it was surprisingly discovered that the extent of levodopa absorption and the peak plasma concentration of certain levodopa and carbidopa oral pharmaceutical dosage forms having an immediate release component and a controlled release component were not affected when administered to a patient under fed or fasting conditions. Such pharmaceutical dosage forms represent a considerable advantage over currently marketed formulations in that they are more convenient and provide more consistent therapy as their use is not constrained by a patient's fed or fasting condition, i.e., a patient may be able to take the formulations of the present invention may be taken with or without food.

The immediate release component comprises a ratio of carbidopa, as a decarboxylase inhibitor, to levodopa of from about 1:1 to about 1:10, inclusive, such that the in vitro dissolution rate of the immediate release component is from about 10% to about 99% levodopa released after 15 minutes and from about 60% to about 99% levodopa released after 1 hour, preferably about 75% to about 99% is released after 1 hour. The controlled release component comprises a ratio of carbidopa to levodopa of from about 1:1 to about 1:10, inclusive, such that the in vitro dissolution rate of the controlled-release component under these conditions is about 10% to about 60% levodopa released after 1 hour; from about 25% to about 80% levodopa released after 2 hours; from about 30% to about 85% levodopa released after 4 hours; and from about 40% to about 99% levodopa released after about 6 hours. The ratio of levodopa in the immediate release component to levodopa in the controlled release component is from about 1:1 to about 1:6, inclusive.

In certain embodiments of the invention, the ratio of carbidopa, as a decarboxylase inhibitor, to levodopa will be from about 1:4 to about 1:10, inclusive. In alternative embodiments, the ratio of carbidopa to levodopa will be from about 1:1 to about 1:3, inclusive.

The formulations of the present invention provide an initial peak plasma level (tₚₑₐₖ₁) of levodopa in vivo between 0.1 and 0.75 hours after administration of the dosage form, preferably between 0.4 and 0.7 hours under fasting conditions. In a preferred embodiment, the ratio of levodopa in the immediate release component to the levodopa in the controlled release component is about 1:1 to about 1:4. In preferred embodiments, the initial peak plasma level of levodopa obtained in vivo occurs between 0.5 and 0.7 hours administration of said dosage form under fasting conditions, the dosage form provides a Cₘₐₓ that is different from Cₚₑₐₖ₁ and wherein the ratio of Cₚₑₐₖ₁ to Cₘₐₓ is from about 0.5 to 1 after administration of the dosage form to a patient under fasting conditions.

The present invention further provides a method of reducing the intra-patient variability of plasma levodopa levels under fed and fasting conditions during carbidopa/levodopa therapy in a patient suffering from a pathology characterized by reduced levels of dopamine in a patient's brain. A method comprises administering to the patient a pharmaceutical dosage form comprising carbidopa and levodopa in a ratio of from about 1:1 to about 1:10, inclusive, wherein the in vitro dissolution rate of the pharmaceutical dosage form according to measurements under the USP paddle method of 50 rpm in 900 ml aqueous buffer at pH 1.2 at 37° C. is from about 20% to about 60% levodopa released after 5 minutes, from about 40% to about 70% released after 30 minutes, and from about 50% to about 80% released after 1 hour.

In a preferred embodiment, the controlled release component is a matrix dosage form comprising a gel-forming polymer to control the release of the decarboxylase inhibitor and levodopa from the dosage form. Preferably, the dosage form is a tablet, and the gel-forming polymer is a cellulose ether, such as hydroxylpropyl cellulose.

### BRIEF DESCRIPTION OF THE DRAWINGS

FIG. 1 is a graph of the dissolution profiles of carbidopa/levodopa immediate release (IR) 25/100 mg formulations PX03002 and PX03102 according to measurements under the USP paddle method of 50 rpm in 900 ml acetate buffer at pH 4 at 37° C.

FIG. 2 is a graph of the dissolution profile of a carbidopa/levodopa controlled release (CR) 50/200 mg formulation PX00502 according to measurements under the USP paddle method of 50 rpm in 900 ml acetate buffer at pH 4 at 37° C.

FIG. 3 is a graph of the dissolution profiles of carbidopa/levodopa 75/300 mg formulations PX03602 and PX04002 according to measurements under the USP paddle method of 50 rpm in 900 ml acetate buffer at pH 4 at 37° C.

FIG. 4 is a graph of the dissolution profiles of carbidopa/levodopa immediate release (IR) 25/100 mg formulations PX00102, PX02001, and SINEMET® according to measurements under the USP paddle method of 50 rpm in 900 ml at pH 1.2(0.1 N HCL) at 37° C.

FIG. 5 is a graph of the dissolution profiles of carbidopa/levodopa controlled release (CR) 50/200 mg formulations PX00302, PX00502, and SINEMET® CR according to measurements under the USP paddle method of 50 rpm in 900 ml at pH 1.2 (0.1 N HCL) at 37° C.

FIG. 6 is a graph of the dissolution profiles of carbidopa/levodopa formulations PX03602 (controlled release, 75/300 mg), PX04002 (controlled release, 75/300 mg), Brand K5370 (SINEMET®, 25/100 mg), and SINEMET® CR (50/200 mg) according to measurements under the USP paddle method of 50 rpm in 900 ml at pH 1.2 (0.1 N HCL) at 37° C.

FIG. 7 is a table showing that ingesting food has substantially no effect on the Cₘₐₓ and AUC of levodopa from a dosage form according to the present invention.

FIG. 8 is a graph of the levodopa plasma concentrations in 18 healthy subjects (range 19-69 years) following a single oral administration of IPX054 200mg, prepared according to Example 9, as well as SINEMET® 25-100, SINEMET® CR 25-100, and SINEMET® CR 50-200 products under fasting conditions.

FIG. 9 is a graph of the concentration (median) versus time profiles of levodopa in healthy subjects (range: 45-75 years) following a single oral administration of IPX054 at different tablet strengths under fasting conditions (N=20).

### DETAILED DESCRIPTION OF THE PREFERRED EMBODIMENTS

The present invention is directed to methods of treating symptoms, pathologies or diseases characterized by reduced levels of dopamine in a patient's brain, including neurological or movement disorders such as restless leg syndrome, Parkinson's disease and secondary Parkinsonism, Huntington's disease, Shy-Drager syndrome and conditions resulting from brain injury including carbon monoxide or manganese intoxication.

For purposes of the present invention the term “controlled release” refers to a pharmaceutical dosage form which releases one or more active pharmaceutical agents over a prolonged period of time, in this case over a period of more than 1 hour. Controlled release (CR) components can also be referred to as sustained release (SR), prolonged release (PR), or extended release (ER). When used in association with the dissolution profiles discussed herein, the term “controlled release” refers to that portion of a dosage form made according to the present invention which delivers active agent over a period of time greater than 1 hour. “Immediate release” refers to a dosage form which releases active agent substantially immediately upon contact with gastric juices and will result in substantially complete dissolution within about 1 hour. Immediate release (IR) components can also be referred to as instant release. When used in association with the dissolution profiles discussed herein, the term “immediate release” refers to that portion of a dosage form made according to the present invention which delivers active agent over a period of time less than 1 hour.

Bioavailability and bioequivalence can be studied by measuring the rate and extent of absorption of an active ingredient. The area under the drug concentration time curve (AUC) indicates the extent of absorption. Cₘₐₓ refers to maximum concentration, and Tₘₐₓ refers to the time of maximum concentration.

Initial peak plasma level refers to the first peak in blood plasma level of active agent (Peak1) and may be followed by one or more additional peaks. The maximum levodopa plasma level in the initial peak is referred to as Cₚₑₐₖ₁. Cₚₑₐₖ₁ or a subsequent peak levodopa plasma level will be Cₘₐₓ. As used herein, “Tₚₑₐₖ₁” refers to the time of the first peak levodopa plasma level, typically obtained within 45 minutes of a single oral administration in healthy adults under fasting conditions.

The USP paddle method refers to the Paddle and Basket Method as described in United States Pharmacopoeia, Edition XXII (1990).

As used herein, the term patient means a human, for which administration of a pharmaceutical dosage form of the present invention would provide therapeutic benefit.

The active agents for use in dosage forms according to the present invention include levodopa and carbidopa their salts, derivatives and pro-drugs. The terms “levodopa” and “carbidopa” are meant to embrace these chemical compounds themselves, pro-drugs thereof, N-oxides thereof, the pharmaceutically acceptable salts thereof, derivatives thereof, and the solvates thereof, e.g. hydrates, where the context so permits. Similarly, reference to intermediates, whether or not they themselves are claimed, is meant to embrace their salts, and solvates, where the context so permits.

The term “derivative” means a chemically modified compound wherein the modification is considered routine by the ordinary skilled chemist, such as an ester or an amide of an acid, protecting groups, such as a benzyl group for an alcohol or thiol, and tert-butoxycarbonyl group for an amine.

The term “effective amount” means an amount of a compound or composition according to the present invention effective in producing the desired therapeutic effect.

The term “analogue” means a compound which comprises a chemically modified form of a specific compound or class thereof, and which maintains the pharmaceutical and/or pharmacological activities characteristic of said compound or class.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quartemary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluensulfonic, methanesulfonic, ethane dislfonic, oxalic, isethionic, and the like.

The phrase “pharmaceutically acceptable” is employed herein to refer to those compounds, materials, compositions; and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The term “about” when used in connection with percentages means ±1%.

The term “pro-drugs”, as the term is used herein, is intended to include any chemical entity which releases or is metabolized into an active drug of the present invention in vivo when such pro-drug is administered to a mammalian subject. Since pro-drugs are known to enhance numerous desirable qualities of pharmaceuticals (i.e., solubility, bioavailability, manufacturing, etc.) the compounds of the present invention may be delivered in pro-drug form. Thus, the present invention is intended to cover pro-drugs of the presently claimed compounds, methods of delivering the same, and compositions containing the same.

One example of a pro-drug for levodopa is 3-hydroxy-L-tyrosine ethyl ester. In the formulations of the present invention, 3-hydroxy-L-tyrosine ethyl ester can be used in combination with levodopa or as a replacement for levodopa in any of the formulations. Generally, an appropriate pro-drug for levodopa can be used in combination with levodopa or used as a replacement for levodopa in any of the levodopa/carbidopa formulations of the present invention. Similarly, an appropriate pro-drug for carbidopa can be used in combination with levodopa or used as a replacement for carbidopa in any of the levodopa/carbidopa formulations of the present invention.

Pro-drugs of the present invention are prepared by modifying functional groups present in the compound in such a way that the modifications are cleaved, either in routine manipulation or in vivo, to the active compound. The transformation in vivo may be, for example, as the result of some metabolic process, such as chemical or enzymatic hydrolysis of a carboxylic, phosphoric or sulphate ester, or reduction or oxidation of a susceptible functionality. Pro-drugs within the scope of the present invention include compounds wherein a hydroxy, amino, or sulfhydryl group is bonded to any group from which it can be cleaved to form a free hydroxyl, free amino, or free sulfydryl group, respectively, when the pro-drug of the present invention is administered to a mammalian subject. Functional groups which may be rapidly transformed by metabolic cleavage in vivo form a class of groups reactive with the carboxyl group of the compounds of this invention. They include, but are not limited to, such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkysilyl (such as trimethyl- and triethysilyl), monoesters formed with dicarboxylic acids (such as succinyl), and the like. Because of the ease with which metabolically cleavable groups of the compounds useful according to this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the compound by virtue of the presence of the metabolically cleavable group.

A thorough discussion of pro-drugs is provided in the following: Design of Pro-drugs, H. Bundgaard, ed., Elsevier, 1985; Methods in Enzymology, K. Widder et al., ed., Academic Press, 42, p. 309-396, 1985; A Textbook of Drug Design and Development, Krogsgaard-Larsen and H. Bundgaard, ed., Chapter 5; “Design and Applications of Pro-drugs” p. 113-191, 1991; Advanced Drug Delivery Reviews, H. Bundgard, 8, p. 1-38, 1992; Journal of Pharmaceutical Sciences, 77, p. 285, 1988; Chem. Pharm. Bull., N. Nakeya et al., 32, p. 692, 1984; Pro-drugs as Novel Delivery Systems, T. Higuchi and V. Stella, Vol. 14 of the A.C.S. Symposium Series, and Bioreversible Carriers in Drug Design, Edward B. Roche, ed., American Pharmaceutical Association and Pergamon Press, 1987, each of which is incorporated herein by reference.

An embodiment of the present invention is directed to a pharmaceutical dosage form having an immediate release component and a controlled release component. The immediate release component comprises a ratio of carbidopa to levodopa from about 1:1 to about 1:50, preferably about 1:1 to about 1:10, such that the in vitro dissolution rate of the immediate release component, according to measurements under the USP paddle method of 50 rpm in 900 ml aqueous buffer at pH 4 at 37° C., is from about 10% to about 99% levodopa released after 15 minutes and from about 75% to about 99% levodopa released after 1 hour. The controlled release component comprises a ratio of carbidopa to levodopa of from about 1:1 to about 1:50 inclusive, preferably about 1:1 to about 1:10 inclusive, such that the in vitro dissolution rate of the controlled release component, according to measurements under the USP paddle method of 50 rpm in 900 ml aqueous buffer at pH 4 at 37° C., is about 10% to about 60% levodopa released after 1 hour; from about 25% to about 80% levodopa released after 2 hours; and from about 40% to about 95% levodopa released after 6 hours.

Because the formulations of the present invention contain both immediate release and controlled release components, the in vivo plasma levodopa levels will generally demonstrate two or more levodopa peaks in most subjects. The initial absorption rate of levodopa in healthy adults after a single dose of under fasting conditions is rapid, with the first peak concentration (Cₚₑₐₖ₁) generally occurring between 0.1 and 0.75 hours, usually between 0.4 and 0.7 hours, which is similar to that of an immediate-release carbidopa-levodopa product and faster than those of extended-release carbidopa-levodopa products.

Maximum plasma concentrations (Cₘₐₓ) of levodopa generally occur between 0.75 and 2.5 hours (Tₘₐₓ) after administration of the dosage forms of the present invention under fasting conditions. Therefore, Cₚₑₐₖ₁ will usually be different from Cₘₐₓ, which occurs at a later time point. Where the amount of levodopa in the immediate-release component is greater than the amount in the controlled-release component, however, Cₚₑₐₖ₁ may be Cₘₐₓ. Carbidopa is generally absorbed more slowly than levodopa, and will typically exhibit a Cₘₐₓ occurring at 2.75 to about 4 hours after administration under fasting conditions.

Total daily dosages of the compounds useful according to this invention administered to a host in single or divided doses are generally in amounts of from about 0.01 mg/kg to about 100 mg/kg body weight daily, and preferably from about 0.05 mg/kg to about 50 mg/kg body weight daily. Both the levodopa and carbidopa doses fall within this mg/kg/day dosage range.

The skilled artisan will appreciate that daily dosages having an amount of active agent sufficient to treat Parkinson's disease will generally contain from about 25 mg to about 4,000 mg of levodopa in combination with from about 5 mg to about 600 mg of carbidopa. Dosage forms according to the present invention may also contain from about 25 or preferably about 100 mg to preferably about 300 or about 600 mg of levodopa in combination with from about 12.5 or preferably about 50 mg to preferably about 75 or about 200 mg of carbidopa. It will be understood, however, that the specific dose level for any particular patient will depend upon a variety of factors including body weight, general health, gender, diet, time and route of administration, rates of absorption and excretion, combination with other drugs, and the severity of the particular disease being treated.

The ratio of immediate release to controlled release levodopa in dosage forms according to an embodiment of the present invention is preferably in the range of about 1:1 to about 1:6 inclusive, most preferably about 1:1 to about 1:4 inclusive. Exemplary dosage forms of the present invention include dosage forms where the immediate release carbidopa/levodopa amounts are 12.5 mg/50 mg carbidopa/levodopa or 25 mg/100 mg carbidopa/levodopa, and the controlled release carbidopa/levodopa amounts are 12.5 mg/50 mg carbidopa/levodopa, 25 mg/100 mg carbidopa/levodopa, and 50 mg/200 mg carbidopa/levodopa. Preferably, the dosage form will contain a ratio of carbidopa to levodopa of about 1:4 to about 1:10 inclusive. While it is not critical that the carbidopa/levodopa ratios be identical in the immediate release and controlled release components, the dosage forms of the present invention will typically include at least about 12.5 mg immediate release carbidopa and an overall carbidopa to levodopa ratio of about 1:1 to about 1:10 inclusive.

Upon administration of these exemplary dosage strengths under fasting conditions, levodopa Cₚₑₐₖ₁ typically will be between 700 and 1500 ng/ml, levodopa Cₘₐₓ typically will be between 750 and 2400 ng/ml, and AUC typically will be between 1400 and 10,000 ng/ml-hr.

The active ingredients of the present inventions, levodopa and carbidopa (an AAAD inhibitor), preferably, are present in a ratio of from about 1:1 to about 1:10 inclusive. Importantly, the present invention provides dosage forms with higher ratios of carbidopa (an AAAD inhibitor) to levodopa than are currently available. In particular, ratios of carbidopa (an AAAD inhibitor) to levodopa in the range of 1:1 to 1:3 are provided. In contrast, commercially available products have proportionally less AAAD inhibitors, in that they contain ratios of 1:4 or 1:10 AAAD inhibitor to levodopa.

Studies suggest that a daily dose for carbidopa of 75 mg to 150 mg per day is required to maximally inhibit systemic dopa decarboxylase. One study, however, suggests that additional carbidopa, even in patients receiving 125 mg to 175 mg doses of carbidopa, can further enhance the bioavailability of levodopa. (Cederbaum, J. M., et al., Clinical Neuropharmacology, 1986, 9(2): 153-159). Another study, however, concluded that additional carbidopa produced no additional benefit. (Contin, M., et al., Clinical Neuropharmacology, 1989, 12(1): 75-81). Taken together, the results of these studies support that optimum doses of carbidopa (or other AAAD inhibitors) vary significantly among individuals who are treated with levodopa.

Low doses of levodopa, in combination with AAAD inhibitors, are increasingly being used to avoid potential long-term complications associated with high doses of levodopa. When the carbidopa-levodopa combination is used in the existing ratios of 1:4 or 1:10, low doses of levodopa may be associated with insufficient inhibition of peripheral decarboxylase activity, leading to inadequate bioavailability of levodopa and/or unwanted side effects such as nausea or vomiting. Patients taking low doses of levodopa would include levodopa-naive patients who require dose titration, patients with mild Parkinsonism who may be stabilized with low and less frequent maintenance levodopa regimens, and patients who are treated with low doses of levodopa-carbidopa in combination with other anti-Parkinson's agents such as dopamine agonists or monoamine oxidase inhibitors.

Therefore, the formulations of the present invention containing carbidopa (a preferred AAAD inhibitor) to levodopa in ratios ranging from 1:1 to 1:3 will provide proportionally more carbidopa (AAAD inhibitor), such that patients taking low doses of levodopa will obtain sufficient daily an AAAD inhibitor to adequately inhibit peripheral decarboxylase activity and improve the bioavailability of levodopa. Ideally, a range of dosages are provided containing fixed-dosage amounts of carbidopa. These dosage forms contain at least about 15 mg, preferably about 25 mg carbidopa. In this aspect of the invention, preferred dosages contain 25 mg carbidopa-25 mg levodopa; 25 mg carbidopa-50 mg levodopa; or 25 mg carbidopa-75 mg levodopa.

The dosage forms of the present invention are designed to administer active agent according to the combination of two release profiles. The first profile is an immediate release burst of carbidopa (a preferred decarboxylase inhibitor) and levodopa to provide early relief from symptoms via quick onset of effective blood plasma levels of active agent. Such early release is such that the in vitro dissolution rate of the immediate release component, according to measurements under the USP paddle method of 50 rpm in 900 ml aqueous buffer at pH 4 at 37° C. are from about 10% to about 99% levodopa released after 15 minutes and from about 75% to about 99% levodopa released after 1 hour.

The second profile is a controlled release profile in which the combination of active ingredients is released slowly over time to provide a plasma level effective to alleviate the symptoms of Parkinson's disease over a prolonged period. This controlled release profile may be over a period of 3, 4, 6, 8 or 12 hours. Furthermore, the controlled release profile of the present invention is such that the in vitro dissolution rate of the controlled release component, according to measurements under the USP paddle method of 50 rpm in 900 ml aqueous buffer at pH 4 at 37° C., are from about 10% to about 60% levodopa released after 1 hour; from about 25% to about 80% released after 2 hours; from about 30% to about 85% levodopa released after 4 hours and from about 40% to about 99% levodopa released after about 6 hours.

In embodiments of the invention comprising preferred ratios of immediate release and controlled release components, the combined dosage form exhibits an in vitro dissolution rate according to measurements under the USP paddle method of 50 rpm in 900 ml aqueous buffer at pH 1.2 at 37° C. of from about 20% to about 60% levodopa released after 5 minutes, from about 40% to about 70% released after 30 minutes, and from about 50% to about 80% released after 1 hour.

The active ingredients of the present invention may be mixed with pharmaceutically acceptable carriers, diluents, adjuvants, excipients, or vehicles, such as preserving agents, fillers, polymers, disintegrating agents, glidants, wetting agents, emulsifying agents, suspending agents, sweetening agents, flavoring agents, perfuming agents, lubricating agents, acidifying agents, and dispensing agents, depending on the nature of the mode of administration and dosage forms. Such ingredients, including pharmaceutically acceptable carriers and excipients that may be used to formulate oral dosage forms, are described in the Handbook of Pharmaceutical Excipients, American Pharmaceutical Association (1986), incorporated herein by reference in its entirety. Examples of pharmaceutically acceptable carriers include water, ethanol, polyols, vegetable oils, fats, waxes polymers, including gel forming and non-gel forming polymers, and suitable mixtures thereof. Examples of excipients include starch, pregelatinized starch, Avicel, lactose, milk sugar, sodium citrate, calcium carbonate, dicalcium phosphate, and lake blend. Examples of disintegrating agents include starch, alginic acids, and certain complex silicates. Examples of lubricants include magnesium stearate, sodium lauryl sulphate, talc, as well as high molecular weight polyethylene glycols. The artisan of ordinary skill in the art will recognize that many different excipients can be used in formulations according to the present invention and the list provided herein is not exhaustive.

Dosage Forms

Dosage forms can be made according to well known methods in the art. Some preferred methods are described below.

Matrix Dosage Forms

The term matrix, as used herein, is given its well known meaning in the pharmaceutical arts as a solid material having an active agent incorporated therein. Upon exposure to a dissolution media, channels are formed in the solid material so that the active agent can escape. Dosage forms according to one embodiment of the present invention may be in the form of coated or uncoated matrices. A coating, for example, may contain immediate release carbidopa alone, or in the alternative, a combination of levodopa and carbidopa, and the matrix itself can contain the controlled release combination of levodopa and carbidopa.

The skilled artisan will appreciate that the matrix material can be chosen from a wide variety of materials which can provide the desired dissolution profiles. Materials can include, for example, one or more gel forming polymers such as polyvinyl alcohol, cellulose ethers including, for example, hydroxypropylalkyl celluloses such as hydroxypropyl methyl cellulose, hydroxyalkyl celluloses such as hydroxypropyl cellulose, natural or synthetic gums such as guar gum, xanthum gum, and alginates, as well as ethyl cellulose, polyvinyl pyrrolidone, fats, waxes, polycarboxylic acids or esters such as the Carbopol R series of polymers, methacrylic acid copolymers, and methacrylate polymers.

Methods of making matrix dosages are well known in the art and any known method of making such dosages which yields the desired immediate release and controlled release dissolution profiles can be used. One such method involves the mixture of the levodopa and carbidopa combination with a solid polymeric material and one or more pharmaceutically acceptable excipients which are then blended and compressed in controlled release tablet cores. Such tablet cores can be used for further processing as bi-layer tablets, press coated tablets, or film coated tablets.

A coating containing the immediate release carbidopa or carbidopa and levodopa in combination can be added to the outside of the controlled release tablet cores to produce a final dosage form. Such a coating can be prepared by mixing carbidopa alone, or a combination of levodopa and carbidopa, with polyvinylpyrrolidone (PVP) 29/32 or hydroxypropyl methylcellulose (HPMC) and water/isopropyl alcohol and triethyl acetate. Such an immediate release coating can be spray coated onto the tablet cores. The immediate release coating may also be applied using a press-coating process with a blend consisting of 80% by weight levodopa and carbidopa and 20% by weight of lactose and hydroxypropyl methylcellulose type 2910. Press coating techniques are known in the art and are described in U.S. Pat. No. 6,372,254 to Ting et al., incorporated herein by reference in its entirety.

In addition, the formulation of respective release components can occur by appropriate granulation methods as is well known in the art. In wet granulation, solutions of the binding agent are added with stirring to the mixed powders. The powder mass is wetted with the binding solution until the mass has the consistency of damp snow or brown sugar. The wet granulated material is forced through a sieving device. Moist material from the milling step is dried by placing it in a temperature controlled container. After drying, the granulated material is reduced in particle size by passing it through a sieving device. Lubricant is added, and the final blend is then compressed into a matrix dosage form.

In fluid-bed granulation, particles of inert material and/or active agent are suspended in a vertical column with a rising air stream. While the particles are suspended, a common granulating material in solution is sprayed into the column. There is a gradual particle buildup under a controlled set of conditions resulting in tablet granulation. Following drying and the addition of lubricant, the granulated material is ready for compression.

In dry-granulation, the active agent, binder, diluent, and lubricant are blended and compressed into tablets. The compressed large tablets are comminuted through the desirable mesh screen by sieving equipment. Additional lubricant is added to the granulated material and blended gently. The material is then compressed into tablets.

In a preferred embodiment the dosage form is a bi-layer tablet prepared by a wet granulation process. In this embodiment, a controlled-release blend is prepared by mixing carbidopa, levodopa and a gel-forming polymer and granulating with purified water. The resulting granulation is then dried, screened, and oversized particles milled through an appropriate sized screen. The screened and milled material is then mixed with a lubricant. Separately, an immediate-release blend is prepared by mixing carbidopa, levodopa, and appropriate excipients with a solution containing a binding agent and granulating. The resulting wet granulation is then dried, screened and milled as described above, and mixed with a lubricant. The final immediate-release and controlled-release blends are then compressed into tablets using a standard rotary tablet press.

Particle Based Dosage Forms

Immediate Release Particles

The immediate release/controlled release dosage forms of the present invention can also take the form of pharmaceutical particles. The dosage forms can include immediate release particles in combination with controlled release particles in a ratio sufficient to deliver the desired dosages of active agents. The controlled release particles can be produced by coating the immediate release particles.

The particles can be produced according to any of a number of well known methods for making particles. The immediate release particles comprise the active agent combination and a disintegrant. Suitable disintegrants include, for example, starch, low-substitution hydroxypropyl cellulose, croscarmellose sodium, calcium carboxymethyl cellulose, hydroxypropyl starch, and microcrystalline cellulose.

In addition to the above-mentioned ingredients, a controlled release matrix may also contain suitable quantities of other materials, for example, diluents, lubricants, binders, granulating aids, colorants, flavorants, and glidants that are conventional in the pharmaceutical arts. The quantities of these additional materials are sufficient to provide the desired effect to the desired formulation. A controlled release matrix incorporating particles may also contain suitable quantities of these other materials such as diluents, lubricants, binders, granulating aids, colorants, flavorants, and glidants that are conventional in the pharmaceutical arts in amounts up to about 75% by weight of the particulate, if desired.

Particles can assume any standard structure known in the pharmaceutical arts. Such structures include, for example, matrix particles, non-pareil cores having a drug layer and active or inactive cores having multiple layers thereon. A controlled release coating can be added to any of these structures to create a controlled release particle.

The term particle as used herein means a granule having a diameter of between about 0.01 mm and about 5.0 mm, preferably between about 0.1 mm and about 2.5 mm, and more preferably between about 0.5 mm and about 2 mm. The skilled artisan will appreciate that particles according to the present invention can be any geometrical shape within this size range and so long as the mean for a statistical distribution of particles falls within the particle sizes enumerated above, they will be considered to fall within the contemplated scope of the present invention.

The release of the therapeutically active agent from the controlled release formulation of the present invention can be further influenced, i.e., adjusted to a desired rate, by the addition of one or more release-modifying agents. The release-modifying agent may be organic or inorganic and include materials that can be dissolved, extracted, or leached from the coating in the environment of use. The pore-forners may comprise one or more hydrophilic materials such as hydroxypropyl methylcellulose. The release-modifying agent may also comprise a semi-permeable polymer. In certain preferred embodiments, the release-modifying agent is selected from hydroxypropyl methylcellulose, lactose, metal stearates, and mixtures thereof.

In one embodiment, oral dosage forms are prepared to include an effective amount of particles as described above within a capsule. For example, melt-extruded particles may be placed in a gelatin capsule in an amount sufficient to provide an effective controlled release dose when ingested and contacted by gastric fluid. In another embodiment, a suitable amount of the particles are compressed into an oral tablet using conventional tableting equipment using standard techniques. Techniques and compositions for making tablets (compressed and molded), capsules (hard and soft gelatin), and pills are also described in Remington's Pharmaceutical Sciences, Arthur Osol, editor, pp. 1553-1593 (1980), incorporated herein by reference. The particles can be made by mixing the relevant ingredients and granulating the mixture. The resulting particles are dried and screened, and the particles having the desired size are used for drug formulation.

Controlled Release Particles

The controlled release particles of the present invention slowly release the combination of levodopa and carbidopa when ingested and exposed to gastric fluids, and then to intestinal fluids. The controlled release profile of the formulations of the present invention can be altered, for example, by increasing or decreasing the thickness of the retardant coating, i.e., by varying the amount of overcoating. The resultant solid controlled release particles may thereafter be placed in a gelatin capsule in an amount sufficient to provide an effective controlled release dose when ingested and contacted by an environmental fluid, e.g., gastric fluid, intestinal fluid or dissolution media. The particles may be overcoated with an aqueous dispersion of a hydrophobic or hydrophilic material to modify the release profile. The aqueous dispersion of hydrophobic material preferably further includes an effective amount of plasticizer, e.g. triethyl citrate. Preformulated aqueous dispersions of ethylcellulose, such as AQUACOAT® or SURELEASE® products, may be used. If a SURELEASE® product is used, it is not necessary to separately add a plasticizer.

The hydrophobic material may be selected from the group consisting of alkylcellulose, acrylic and methacrylic acid polymers and copolymers, shellac, zein, hydrogenated castor oil, hydrogenated vegetable oil, or mixtures thereof. In certain preferred embodiments, the hydrophobic material is a pharmaceutically acceptable acrylic polymer, including but not limited to acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylicacid alkylamine copolymer, poly(methyl methacrylate), poly(methacrylic acid anhydride), polymethacrylate, polyacrylamide, poly(methacrylic acid anhydride), and glycidyl methacrylate copolymers. In alternate embodiments, the hydrophobic material is selected from materials such as one or more hydroxyalkyl celluloses such as hydroxypropyl methycellulose. The hydroxyalkyl cellulose is preferably a hydroxy (C₁ to C₆) alkyl cellulose, such as hydroxypropylcellulose, hydroxypropylmethylcellulose, or preferably hydroxyethylcellulose. The amount of the hydroxyalkyl cellulose in the present oral dosage form is determined, in part, by the precise rate of active agents desired and may vary from about 1% to about 80%.

In embodiments of the present invention where the coating comprises an aqueous dispersion of a hydrophobic polymer, the inclusion of an effective amount of a plasticizer in the aqueous dispersion of hydrophobic polymer can further improve the physical properties of the film. For example, because ethylcellulose has a relatively high glass transition temperature and does not form flexible films under normal coating conditions, it is necessary to plasticize the ethylcellulose before using it as a coating material. Generally, the amount of plasticizer included in a coating solution is based on the concentration of the film-former, e.g., most often from about I percent to about 50 percent by weight of the film-former. Concentration of the plasticizer, however, is preferably determined after careful experimentation with the particular coating solution and method of application.

Examples of suitable plasticizers for ethylcellulose include water-insoluble plasticizers such as dibutyl sebacate, diethyl phthalate, triethyl citrate, tributyl citrate, and triacetin, although other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to, citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as EUDRAGIT® RL/RS lacquer solutions include polyethylene glycols, propylene glycol, diethyl phthalate, castor oil, and triacetin. Triethyl citrate is an especially preferred plasticizer for aqueous dispersions of ethyl cellulose. It has further been found that addition of a small amount of talc reduces the tendency of the aqueous dispersion to stick during processing and acts a polishing agent.

One commercially available aqueous dispersion of ethylcellulose is the AQUACOAT® product which is prepared by dissolving the ethylcellulose in a water-immiscible organic solvent and then emulsifying the ethylcellulose in water in the presence of a surfactant and a stabilizer. After homogenization to generate submicron droplets, the organic solvent is evaporated under vacuum to form a pseudolatex. The plasticizer is not incorporated into the pseudolatex during the manufacturing phase. Thus, prior to using the pseudolatex as a coating, the AQUACOAT® product is mixed with a suitable plasticizer.

Another aqueous dispersion of ethylcellulose is commercially available as SURELEASE® product (Colorcon, Inc., West Point, Pa., U.S.A.). This product is prepared by incorporating plasticizer into the dispersion during the manufacturing process. A hot melt of a polymer, plasticizer (dibutyl sebacate), and stabilizer (oleic acid) is prepared as a homogeneous mixture which is then diluted with an alkaline solution to obtain an aqueous dispersion which can be applied directly onto substrates.

In one embodiment, the acrylic coating is an acrylic resin lacquer used in the form of an aqueous dispersion, such as that which is commercially available from Rohm Pharma under the trade name EUDRAGIT®. In additional embodiments, the acrylic coating comprises a mixture of two acrylic resin lacquers commercially available from Rohm Pharma under the trade names EUDRAGIT® RL 30 D and EUDRAGIT® RS 30 D. EUDRAGIT® RL 30 D and EUDRAGIT® RS 30 are copolymers of acrylic and methacrylic esters with a low content of quaternary ammonium groups, the molar ratio of ammonium groups to the remaining neutral (meth)acrylic esters being 1:20 in EUDRAGIT® RL 30 and 1:40 in EUDRAGIT® RS 30 D. The mean molecular weight is about 150,000 Daltons. The code designations RL (high permeability) and RS (low permeability) refer to the permeability properties of these agents. EUDRAGIT® RL/RS mixtures are insoluble in water and in digestive fluids; however, coatings formed from them are swellable and permeable in aqueous solutions and digestive fluids.

The EUDRAGIT® RL/RS dispersions may be mixed together in any desired ratio in order to ultimately obtain a controlled-release formulation having a desirable dissolution profile. Desirable controlled-release formulations may be obtained, for instance, from a retardant coating derived from one of a variety of coating combinations, such as 100% EUDRAGIT® RL; 50% EUDRAGIT® RL and 50% EUDRAGIT® RS; or 10% EUDRAGIT® RL and EUDRAGIT® 90 % RS. Of course, one skilled in the art will recognize that other acrylic polymers may also be used, for example, others under the EUDRAGIT® brand. In addition to modifying the dissolution profile by altering the relative amounts of different acrylic resin lacquers, the dissolution profile of the ultimate product may also be modified, for example, by increasing or decreasing the thickness of the retardant coating.

The stabilized product may be obtained by subjecting the coated substrate to oven curing at a temperature above the Tg (glass transition temperature) of the plasticized acrylic polymer for the required time period, the optimum values for temperature and time for the particular formulation being determined experimentally. In certain embodiments of the present invention, the stabilized product is obtained via an oven curing conducted at a temperature of about 45 ° C. for a time period from about 1 to about 48 hours. It is also contemplated that certain products coated with the controlled-release coating of the present invention may require a curing time longer than 24 to 48 hours, e.g., from about 48 to about 60 hours or more.

The coating solutions preferably contain, in addition to the film-former, plasticizer, and solvent system (i.e., water), a colorant to provide elegance and product distinction. Color may be added to the solution of the therapeutically active agent instead of, or in addition to the aqueous dispersion of hydrophobic material. For example, color may be added to an AQUACOAT® product via the use of alcohol or propylene glycol based color dispersions, milled aluminum lakes and opacifiers such as titanium dioxide by adding color with shear to the water soluble polymer solution and then using low shear to the plasticized AQUACOAT® product. Alternatively, any suitable method of providing color to the formulations of the present invention may be used. Suitable ingredients for providing color to the formulation when an aqueous dispersion of an acrylic polymer is used include titanium dioxide and color pigments, such as iron oxide pigments. The incorporation of pigments, may, however, increase the retardant effect of the coating.

Spheroids or beads coated with the therapeutically active agents can be prepared, for example, by dissolving the therapeutically active agents in water and then spraying the solution onto a substrate, for example, non pareil 18/20 beads, using a Wuster insert. Optionally, additional ingredients are also added prior to coating the beads in order to assist the binding of the active agents to the beads, and/or to color the solution, etc. For example, a product which includes hydroxypropyl methycellulose with or without colorant (e.g., OPADRY® product, commercially available from Coloron, Inc.) may be added to the solution and the solution mixed (e.g., for about 1 hour) prior to application onto the beads. The resultant coated substrate, beads in this example, may then be optionally overcoated with a barrier agent to separate the therapeutically active agent from the hydrophobic controlled release coating. An example of a suitable barrier agent is one which comprises hydroxypropylmethylcellulose. However, any film-former known in the art may be used. It is preferred that the barrier agent does not affect the dissolution rate of the final product.

Immediate release particles according to the present invention may be coated with a controlled release coating in order to change the release rate to obtain the dissolution rates according to the present invention.

Press Coated, Pulsatile Dosage Form

In another embodiment of the present invention, the carbidopa and levodopa combination is administered via a press coated pulsatile drug delivery system suitable for oral administration with a controlled release component, which contains a compressed blend of an active agent and one or more polymers, substantially enveloped by an immediate release component, which contains a compressed blend of the active agent and hydrophilic and hydrophobic polymers. The immediate-release component preferably comprises a compressed blend of active agent and one or more polymers with disintegration characteristics such that the polymers disintegrate rapidly upon exposure to the aqueous medium.

The controlled-release component preferably comprises a combination of hydrophilic and hydrophobic polymers. In this embodiment, once administered, the hydrophilic polymer dissolves away to weaken the structure of the controlled-release component, and the hydrophobic polymer retards the water penetration and helps to maintain the shape of the drug delivery system.

In accordance with the present invention, the term “polymer” includes single or multiple polymeric substances, which can swell, gel, degrade or erode on contact with an aqueous environment (e.g., water). Examples include alginic acid, carboxymethylcellulose calcium, carboxymethylcellulose sodium, colloidal silicon dioxide, croscarmellose sodium, crospovidone, guar gum, magnesium aluminum silicate, methylcellulose, microcrystalline cellulose, polacrilin potassium, powdered cellulose, pregelatinized starch, sodium alginate, sodium starch glycolate, starch, ethylcellulose, gelatin, hydroxyethyl cellulose, hydroxypropyl cellulose, hydroxypropyl methylcellulose, polymethacrylates, povidone, pregelaiinized starch, shellac, and zein, and combinations thereof.

The term “hydrophilic polymers” as used herein includes one or more of carboxymethylcellulose, natural gums such as guar gum or gum acacia, gum tragacanth, or gum xanthan, hydroxyethyl cellulose, hydroxypropyl cellulose, hydroxypropyl methylcellulose, methylcellulose, and povidone, of which hydroxypropyl methylcellulose is further preferred. The term “hydrophilic polymers” can also include sodium carboxymethycellulose, hydroxymethyl cellulose, polyethelene oxide, hydroxyethyl methyl cellulose, carboxypolymethylene, polyethelene glycol, alginic acid, gelatin, polyvinyl alcohol, polyvinylpyrrolidones, polyacrylamides, polymethacrylamides, polyphosphazines, polyoxazolidines, poly(hydroxyalkylcarboxylic acids), an alkali metal or alkaline earth metal, carageenate alginates, ammonium alginate, sodium alganate, or mixtures thereof.

The hydrophobic polymer of the drug delivery system can be any hydrophobic polymer which will achieve the goals of the present invention including, but not limited to, one or more polymers selected from carbomer, camauba wax, ethylcellulose, glyceryl palmitostearate, hydrogenated castor oil, hydrogenated vegetable oil type 1, microcrystalline wax, polacrilin potassium, polymethacrylates, or stearic acid, of which hydrogenated vegetable oil type 1 is preferred. Hydrophobic polymers can include, for example, a pharmaceutically acceptable acrylic polymer, including, but not limited to, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methyl methacrylate) copolymer, polyacrylamide, aminoalkyl methacrylate copolymer, poly(methacrylic acid anhydride), and glycidyl methacrylate copolymers. Additionally, the acrylic polymers may be cationic, anionic, or non-ionic polymers and may be acrylates, methacrylates, formed of methacrylic acid or methacrylic acid esters. The polymers may also be pH dependent.

The present invention also provides a method for preparing a press coated, pulsatile drug delivery system suitable for oral administration. This method includes the steps of combining an effective amount of an active agent, or a pharmaceutically acceptable salt thereof, and a polymer to form an immediate-release component; combining an effective amount of an active agent, or a pharmaceutically acceptable salt thereof, and a combination of hydrophilic and hydrophobic polymers to form a controlled release component; and press coating the controlled-release component to substantially envelop the immediate release component.

A preferred embodiment further includes the steps of combining an effective amount of an active agent, or a pharmaceutically acceptable salt thereof, and a polymer to form an immediate release component, and press coating the immediate release component to substantially envelop the controlled release component. In another preferred embodiment, the combining steps can be done by blending, wet granulation, fluid-bed granulation, or dry granulation according to methods recognized in the art.

The term “substantially envelop” is intended to define the total or near-total enclosure of a component. Such an enclosure includes, preferably, at least 80% enclosure, more preferably at least 90% enclosure, and most preferably at least 99% enclosure.

Levodopa absorption in a patient results from a series of factors, including whether the dosage form is administered under fasting or non-fasting conditions. It would improve dosing convenience to provide a dosage form capable of providing at least substantially the same bioavailability of levodopa regardless of whether the administration is under fasting or non-fasting conditions. A food effect is commonly associated with other commercial forms of carbidopa/levodopa dosage forms. Exemplary literature references showing that the SINEMET® and SINEMET® CR brand products can exhibit a food effect include: Kurlan, et al., Erratic Gastric Emptying of Levodopa May Cause Random “Fluctuations” of Parkinsonian Mobility, Neurology, 38:419-421 (1998); Baruzzi et al., Infuence of Meal Ingestion Time on Pharmacokinetics of Orally Administered Levodopa in Parkinsonian Patients, Clinical NeuropharnacologyP, 10:527-537 (1987); Yeh et al., Pharmacokinetics and Bioavailability of SINEMET® SR, Neurology, 39 (Supp. 2):25-38 (1989); Goetz et al., Parkinson's Disease and Motor Fluctuations: Long-acting Carbidopa/Levodopa (CR-4-SINEMET®), Neurology, 37:875-878 (1987); SINEMET® (carbidopa/levodopa) Package Insert, Merck and Bristol-Myers Squibb (2002); and SINEMET® CR (carbidopa/levodopa) Package Insert, Merck & Co., Inc. and Bristol-Myers Squibb Company (2002), the disclosures of which are incorporated herein by reference.

In certain conventional dosage forms, the absorption of levodopa on an empty stomach may be rapid in a patient, whereas meal ingestion before levodopa dosing (i.e., non-fasting conditions) can result in a significant reduction in levodopa absorption, which can delay the time of peak of levodopa concentration in the patient's plasma (Tₘₐₓ) and reduce the peak levodopa plasma level (Cₘₐₓ). For instance, in the Baruzzi et al study, a significant lowering in peak levodopa concentration after meals (non-fasting conditions) on average was reported. Other known dosage forms, such as SINEMET® CR brand products, can show a significant increase in the bioavailability of levodopa in a patient when taken with food as compared to fasting conditions.

As used herein, the expression “no food effect” (or comparable expression) means that the bioavailability of levodopa from a oral pharmaceutical dosage form of the present invention in a patient in the fed state does not differ under FDA standards from the bioavailability in a fasting state. The presence or absence of the food effect may be quantified by making Area Under the Curve (AUC) and maximum plasma concentration (Cₘₐₓ ) measurements according to methods known in the art.

For instance, AUC and Cₘₐₓ measurements may be made by administering the pharmaceutical dosage form to a subject and taking timed blood samples and plotting the serum concentration of levodopa against time. The values obtained represent a number of values taken from the subject(s) across patient populations and are consequently expressed as mean values expressed over the patient population. A comparison of the AUC and Cₘₐₓ values can determine whether or not a food effect is exhibited. In quantitative terms, a pharmaceutical dosage form according to the invention may be said to exhibit no food effect if the 90% confidence interval (CI) for the ratio of means (based on log transformed data) of fed to fasted treatments fall within the interval of 80% to 125% of the AUC_{fed/fasting} and C_{max(fed/fasting)}. Thus, for example, the 90% confidence interval limits for the mean of the AUC in a patient between administrations under fed conditions must fall between 80 and 125% of the values obtained under fasting conditions.

A pharmaceutical dosage form according to the invention can therefore provide reduced variability in bioavailability on an intra-patient basis as well as between individuals. A related aspect of the invention therefore includes a method of reducing intra-patient variability of bioavailability levels of levodopa in patients during oral levodopa/carbidopa therapy, which method comprises administering a pharmaceutical dosage form as described herein which shows no food effect when administered to a patient indiscriminately in the fed or fasting state.

Pharmaceutical dosage forms according to the present invention surprisingly exhibited no food effect, or at least substantially avoided the food effect, as demonstrated in randomized, single-dose, two-way crossover study under fasting and fed conditions in healthy volunteers. The study evaluated the effect of a standardized, high-fat, high-calorie meal on the rate and extent of absorption of a single dose of a bilayer tablet according to the present invention (designated “IPX054“) that contained 75 mg carbidopa and 300 mg levodopa (25 mg carbidopa and 100 mg levodopa in the immediate release component and 50 mg carbidopa and 200 mg levodopa in the controlled release component). This dosage is representative of the other dosage amounts and ratios of active ingredients in a solid dosage form according to the present invention. Each human subject received one dose in each of two dosing periods (total of two doses per subject), with a washout period of seven days between doses. Those subjects randomized into the fed regimen were fed 30 minutes before dosing a standardized high-fat, high-calorie breakfast consisting of: two eggs fried in butter, two slices of toast with butter, eight fluid ounces of whole milk, two strips of bacon and four ounces of hash brown potatoes (standardized FDA high fat meal). Samples were collected within one hour prior to dosing (0 hour) and after dose administration at: 0.12, 0.25, 0.5, 0.75, 1, 1.5, 2, 2.5, 3, 3.5, 4, 4.5, 5, 6, 7, 8, 10 and 12 hours.

The results show that food has no effect on the bioavailabilty of levodopa from a pharmaceutical dosage form according to the present invention, (bilayer tablets designated “IPX054“), as seen in Table 1 (Pharmacokinetic Parameters) and Table 2 (Bioequivalence) hereinbelow. Although there was a slight reduction in absorption initially, the high-calorie and high-fat meal had no effect on the levodopa Cₘₐₓ or AUC values for IPX054. In contrast, the high-calorie, high-fat meal shortens the Tₘₐₓ of carbidopa from about 3.5 to 3 hours and reduces the carbidopa Cₘₐₓ and AUC values of by approximately 50%. Surprisingly, the reduction in carbidopa AUC and Cₘₐₓ levels did not affect the extent of levodopa absorption.

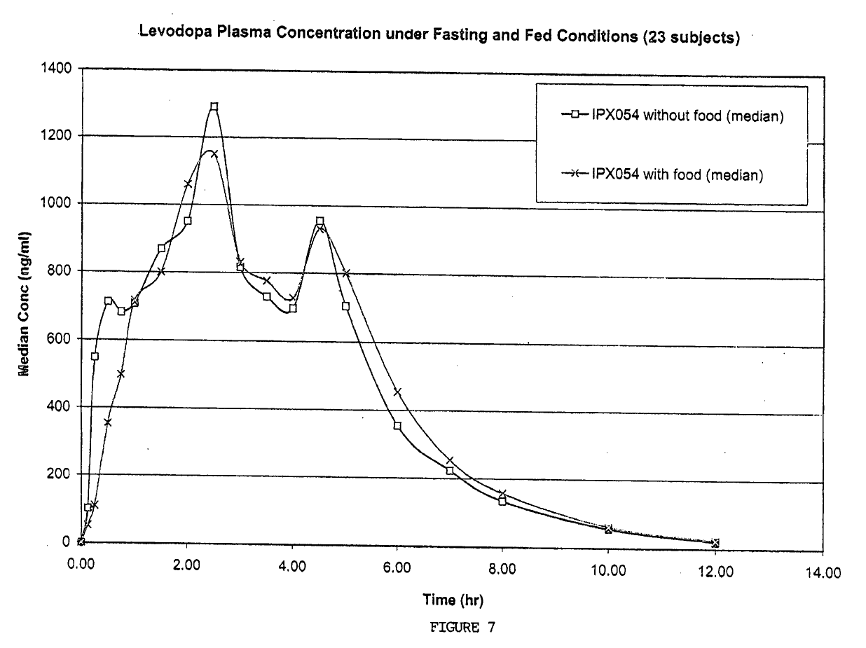

The present invention therefore provides a method of treating a patient suffering from a pathology or disease characterized by reduced levels of dopamine in a patient's brain, such as Parkinson's disease, comprising administering a dosage form according to the invention to a patient in need of such treatment under non-fasting conditions without the adverse food effect on levodopa bioavailability. As seen from FIG. 7, a dosage form of the present invention can provide substantially the same levodopa blood plasma concentration whether the administration is under non-fasting or fasting conditions.

A dosage form according to the present invention is capable of being administered to a patient in need of such treatment to attain comparable peak levodopa blood plasma concentrations regardless of whether the administration is under fasting or non-fasting conditions. The levodopa bioavailability under fasting versus non-fasting conditions when measured as AUC is substantially the same for a given dosage form according to the present invention under fasting and non-fasting conditions. A dosage form according to the present invention is therefore capable of providing consistent therapy to a patient in need of treatment whether under fasting and under non-fasting conditions.

TABLE 1
Pharmacokinetic Parameters
Levodopa (LD) and Carbidopa (CD)

| | | | |
|---|---|---|---|
| PK | Cₘₐₓ | Tₘₐₓ^{a} | AUC_{0-∞} |
| Parameter | (ng/mL) | (hr) | (ng/mL · hr) |

LD

| | | | |
|---|---|---|---|
| Fasting | 1762 (34%) | 0.25-4.5 (2.5) | 6040 (28%) |
| Fed | 1612 (29%) | 0.75-6 (2.5) | 6024 (28%) |

CD

| | | | |
|---|---|---|---|
| Fasting | 282 (42%) | 2-5 (3.5) | 1457 (41%) |
| Fed | 149 (29%) | 1.5-6 (3) | 777 (25%) |

The Tₘₐₓ values in Table 1 are expressed as the range, and median values are shown in parentheses. Cₘₐₓ and AUC values in Table 1 are the means, with the coefficients of variation.

TABLE 2
Bioequivalence Assessment
(without food vs. with food)

| N = 23* | Cₘₐₓ | AUC_{0-∞} |
|---|---|---|
| LD Ratio (Fed/Fasting) | 0.93 | 1.00 |
| 90% Confidence Interval | 0.83-1.03 | 0.92-1.09 |
| CD Ratio (Fed/Fasting) | 0.55 | 0.56 |
| 90% Confidence Interval | 0.49-0.62 | 0.50-0.62 |

*excluding one dropout subject

Tₘₐₓ means the time point at which the serum levels reach their peak; Cₘₐₓ means the serum level attained at the peak. AUCₗₐₛₜ means the area under the curve from time 0 to the last blood-draw time point; and AUC_{0-∞} means the Area Under the Curve from time 0 to infinity, calculated as the sum of the AUCₗₐₛₜ plus Cₗₐₛₜ (last measurable concentration) divided by the elimination rate constant. A comparison of the two values should be nearly identical to confirm that appropriate time points were sampled to characterize the terminal phase of the curve (i.e., elimination phase of the drug in the body). In the example reported, the ratio of fed to fasting AUC (log-transformed data) is calculated as about 1.0, with upper and lower limit 90% confidence levels and the InCₘₐₓ ratio observed for fed/fasting satisfies the FDA criteria for bioequivalence, all as indicated in Table 2.

The following examples describe and illustrate the processes and products of the present invention. These examples are intended to be merely illustrative of the present invention, and not limiting thereof in either scope or spirit. Those skilled in the art will readily understand that variations of the materials, conditions, and processes described in these examples can be used. All references cited herein are incorporated by reference.

### EXAMPLE 1

The method described below was employed to obtain a press coated, pulsatile drug delivery system, the composition of which is set forth in Tables 3 and 4.

Appropriate weights of levodopa and carbidopa (weights shown in Tables 1 and 2) are intimately mixed for use in preparing immediate release and controlled release components of the formulations of the present invention.

Immediate-Release Component

The active agents are first mixed with silicon dioxide in a Patterson-Kelley V-blender for 10 minutes. Then microcrystalline cellulose and croscarmellose sodium are added and blended for 10 more minutes. Finally, magnesium stearate is added to the blender and mixed for another 10 minutes. The powder blend is then compressed using a Manesty Dry-cota with a 0.2031 inch diameter, round, flat-face punch and die set. The hardness of the tablets is maintained at 4±2 kp.

Immediate-Release Component Plus Controlled-Release Component

The active agents are first mixed with silicon dioxide in a Patterson-Kelley V-blender for 10 minutes. Then hydroxypropyl methylcellulose 2208 and microcrystalline cellulose are added and blended for 10 more minutes. Finally, hydrogenated vegetable oil and magnesium stearate are added to the blender and mixed for another 10 minutes. The core tablets are press-coated using the Manesty Dry-cota with 0.3600 inch diameter, round, shallow concave punch and die set. The hardness of the tablets is maintained at 12±4 kp.

### EXAMPLE 2

Immediate-Release Component Plus Controlled-Release Component Plus Immediate-Release Component

The method of manufacture for the controlled-release tablets is the same as described in Example 1. The application of the immediate-release component was done by charging the controlled-release tablets into a perforated pan coater or a fluidized particle coater and coating the tablet cores with a solution consisting of levodopa and carbidopa 80% w/lactose and hydroxypropyl methylcellulose type 2910.

TABLE 3

| | Quantity/Tablet | | | |
|---|---|---|---|---|
| | Example #1 | | Example #2 | |
| | RT-010 (press- | | RT-011 (press | |
| | coated w/o instant- | | coated w/instant- | |
| | release coating) | | release coating) | |
| Immediate-Release (IR) | | | | |
| Compartment | | | | |
| Levodopa/carbidopa 4:1 | 50.0 | mg | 50.0 | mg |
| ratio 80% w/lactose | | | | |
| Croscarmellose sodium | 1.6 | mg | 1.6 | mg |
| Microcrystalline cellulose | 26.8 | mg | 26.8 | mg |
| Colloidal silicon dioxide | 0.8 | mg | 0.8 | mg |
| Magnesium stearate | 0.8 | mg | 0.8 | mg |
| Total: | 80.0 | mg | 80.0 | mg |
| IR Compartment Plus Extended- | | | | |
| Release (ER) Compartment | | | | |
| IR Compartment | 80.0 | mg | 80.0 | mg |
| Levodopa/carbidopa 4:1 | 37.5 | mg | 18.8 | mg |
| ratio 80% w/lactose | | | | |
| Hydroxypropyl | 61.6 | mg | 61.6 | mg |
| methylcellulose type 2208 | | | | |
| Microcrystalline cellulose | 70.3 | mg | 89.0 | mg |
| Hydrogenated vegetable | 46.2 | mg | 46.2 | mg |
| oil type 1 | | | | |
| Colloidal silicon dioxide | 2.2 | mg | 2.2 | mg |
| Magnesium stearate | 2.2 | mg | 2.2 | mg |
| Total: | 300.0 | mg | 300.00 | mg |
| IR Compartment Plus ER | | | | |
| Compartment Plus | | | | |
| Instant-Release Compartment | | | | |
| IR Compartment Plus ER | | | 300.0 | mg |
| Compartment | | | | |
| Levodopa/carbidopa | | | 18.7 | mg |
| 4:1 ratio 80% w/lactose | | | | |
| Hydroxypropyl | | | 1.9 | mg |
| methylcellulose type 2910 | | | | |
| Total: | | | 320.6 | mg |

TABLE 4
EXCIPIENT RANGE

| | Quantity/tablet | | | |
|---|---|---|---|---|
| | Example #1 | | | |
| | RT-010 (press | | | |
| | coated w/o | | | |
| | instant-release | | | |
| | coating) | | Percent | Range |
| Immediate-Release | | | | |
| Compartment | | | | |
| Levodopa/carbidopa | 50.0 | mg | 62.5% | |
| 4:1 ratio 80% w/lactose | | | | |
| Croscarmellose sodium | 1.6 | mg | 2.0% | 0.5-10.0% |
| Microcrystalline cellulose | 26.8 | mg | 33.5% | 18.0-36.0% |
| Colloidal silicon dioxide | 0.8 | mg | 1.0% | 0.5-2.0% |
| Magnesium stearate | 0.8 | mg | 1.0% | 0.5-2.0% |
| Total: | 80.0 | mg | | |
| Extended-Release | | | | |
| Compartment | | | | |
| Levodopa/carbidopa | 37.5 | mg | 17.0% | |
| 4:1 ratio 80% w/lactose | | | | |
| Hydroxypropyl | 61.6 | mg | 28.0% | 15.0-40.0% |
| methylcellulose type 2208 | | | | |
| Microcrystalline cellulose | 70.3 | mg | 32.0% | 8.0-57.0% |
| Hydrogenated vegetable | 46.2 | mg | 21.0% | 10.0-30.0% |
| oil type 1 | | | | |
| Colloidal silicon dioxide | 2.2 | mg | 1.0% | 0.5-2.0% |
| Magnesium stearate | 2.2 | mg | 1.0% | 0.5-2.0% |
| Total: | 220.0 | mg | | |

### EXAMPLE 3

Example 3 employs the ingredients and amounts listed in Tables 5A, 5B, and 5C below for the formulations PX00502, PX03002, and PX03102, respectively.

For each batch, whether PX00502, PX03002 or PX03102, the following procedure is used: All ingredients, except magnesium stearate are weighed and mixed thoroughly. The mixed ingredients are added to a high shear granulator and mixed for 5 minutes, with an impeller speed of 5 and a chopper speed of 4. Deionized water is employed as the granulating agent. Granules so made are dried in an oven overnight and then screened through a #20 mesh (US standard). Oversize granules are milled, screened with the process repeated until all particles can be screened through a #20 mesh. The magnesium stearate is added to the screened particles and mixed thoroughly. The resulting mixture can then be used for different types of dosage forms, such as set out in Example 4.

TABLE 5A

per tablet

| CR PX00502 | (w/w)% | amount |
|---|---|---|
| Carbidopa | 18 | 53.8 |
| Levodopa | 67 | 200.1 |
| Klucel | 12.9 | 38.5 |
| Lake blend | 0.3 | 0.9 |
| Mg stearate | 1.8 | 5.4 |
| Total | 100 | 298.7 |

TABLE 5B

per tablet

| PX03002 | (w/w)% | amount |
|---|---|---|
| Carbidopa | 11.3 | 27 |
| Levodopa | 41.9 | 100 |
| Avicel | 33.2 | 79.2 |
| Starch | 11.1 | 26.5 |
| Acdisol | 0.8 | 1.9 |
| Mg stearate | 1.7 | 3.8 |
| Total | 100 | 238.4 |

TABLE 5C

per tablet

| PX03102 | (w/w)% | amount |
|---|---|---|
| Carbidopa | 9.3 | 26.9 |
| Levodopa | 34.6 | 100.1 |
| Avicel | 27.4 | 79.3 |
| Starch | 27.4 | 79.3 |
| Mg Stearate | 1.3 | 3.8 |
| Total | 100 | 289.4 |

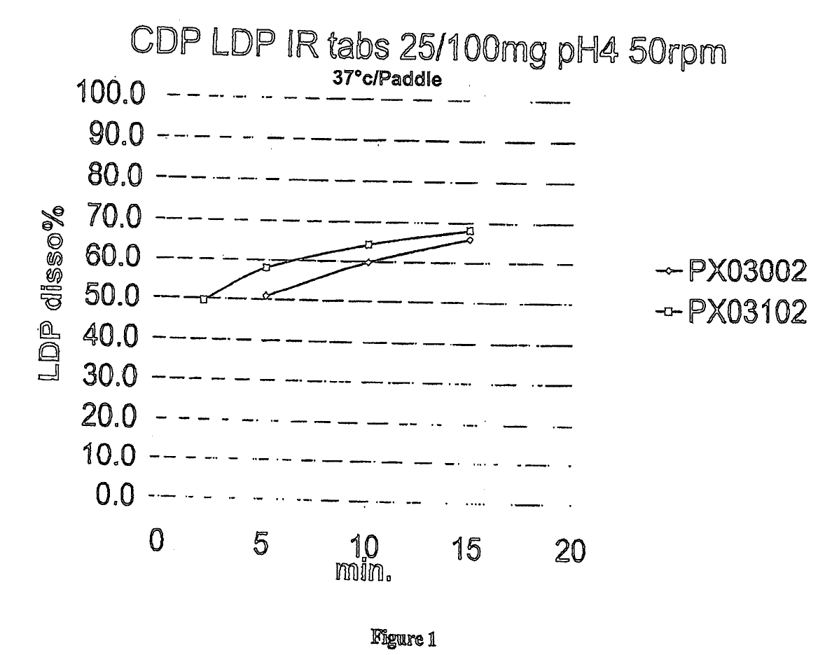

FIG. 1 shows the dissolution profiles of profiles of carbidopa/levodopa immediate release (IR) 25/100 mg formulations PX03002 and PX03102 (Tables 5B and 5C). As discussed above, all dissolution profiles were carried out by the standard USP paddle method of 50 rpm in 900 ml aqueous buffer at pH 4 at 37° C.

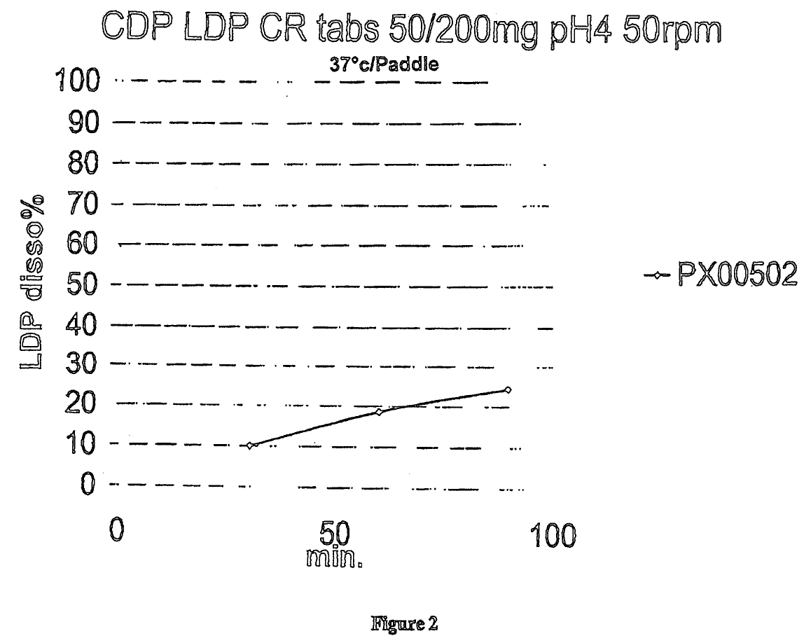

FIG. 2 shows the dissolution profile of a carbidopa/levodopa controlled release (CR) 50/200 mg formulation PX00502 (Table 5A).

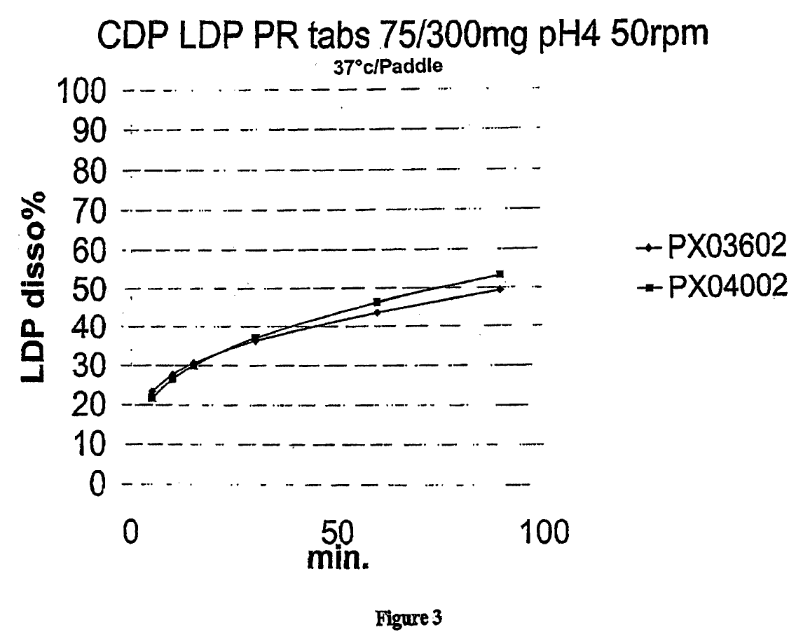

FIG. 3 shows the dissolution profiles of carbidopa/levodopa 75/300 mg formulations PX03602 and PX04002. Note that controlled release (or prolonged release (PR)) tablets PX03602 comprise the combination of PX00502(CR) and PX03102, and PR tablets PX04002 comprise the combination of PX00502(CR) and PX03002.

### EXAMPLE 4

The particles produced in Example 3 are segregated into two equal portions of 125 grams each. One portion is coated in a fluidized pan with a mixture of 24.25 g of PVP 29/32, 1000 g of deionized water and isopropyl alcohol (15%), and 0.75 g of triethyl acetate. The particles are dried and thoroughly mixed with the uncoated particles. The particle mixture is then loaded into immediate release gelatin capsules.

### EXAMPLE 5

Particles produced according to lots PX03002 and PX00502 of Example 3 are loaded into the two separate hoppers of a dual layer tablet punch. The punch is actuated and two-layer tablets are produced.

### EXAMPLE 6

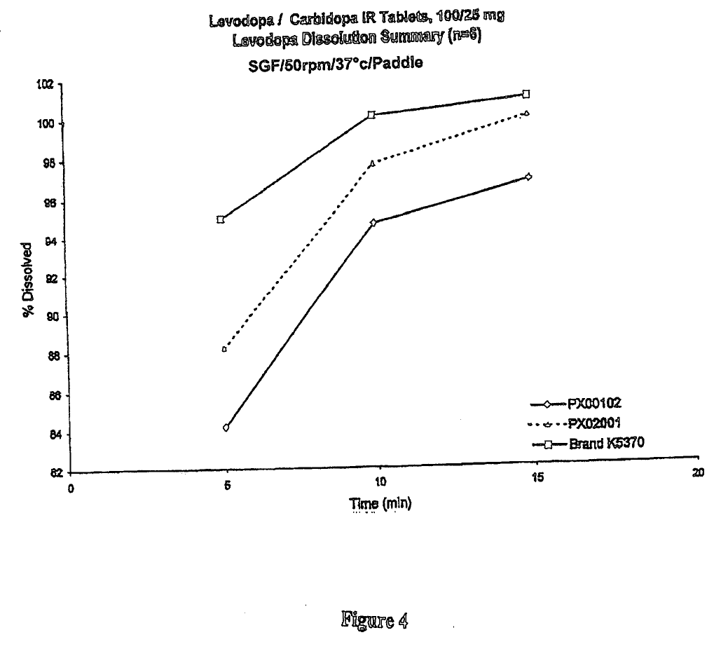

The dissolution summaries for carbidopa/levodopa immediate release (IR) 25/100 mg formulations PX00102, PX02001, and SINEMET® (25/100 mg) are shown in Tables 6, 7, and 8, respectively. All data was obtained according to measurements under the USP paddle method of 50 rpm in 900 ml at pH 1.2 (0.1 N HCL) at 37° C. FIG. 4 is a graph of the dissolution profiles of carbidopa/levodopa immediate release (IR) 25/100 mg formulations PX00102, PX02001, and SINEMET®.

The formulations PX00102 and PX02001 were prepared by granulating carbidopa, levodopa, microcrystalline cellulose, starch, and croscarmellose sodium in a high-shear granulator using a povidone aqueous solution. Granules were dried in an oven at 60±5° C. and screened through USP 25 mesh screen. Oversized granules were then milled through USP 16 mesh screen. Screened and milled granules were then blended with Magnesium Stearate. The blend was then compressed into tablets using a rotary tablet press.

Quantitative Composition of25-100 mg CD-LD IR Tablets, Lot PX00102

| | Amount (mg) |
|---|---|
| Components | per tablet |
| Carbidopa | 27 |
| Levodopa | 100 |
| Microcrystalline | 85 |
| Cellulose | |
| Starch | 26 |
| Povidone | 26 |
| Croscarmellose | 5 |
| Sodium | |
| Magnesium | 4 |
| Stearate | |
| Total | 273 |

Quantitative Composition of 25-100 mg CD-LD IR Tablets, Lot PX02001

| | Amount (mg) |
|---|---|
| Components | per tablet |
| Carbidopa | 27 |
| Levodopa | 100 |
| Microcrystalline | 79 |
| Cellulose | |
| Starch | 26 |
| Povidone | 20 |
| Croscarmellose | 8 |
| Sodium | |
| Magnesium | 4 |
| Stearate | |
| Total | 264 |

### EXAMPLE 7

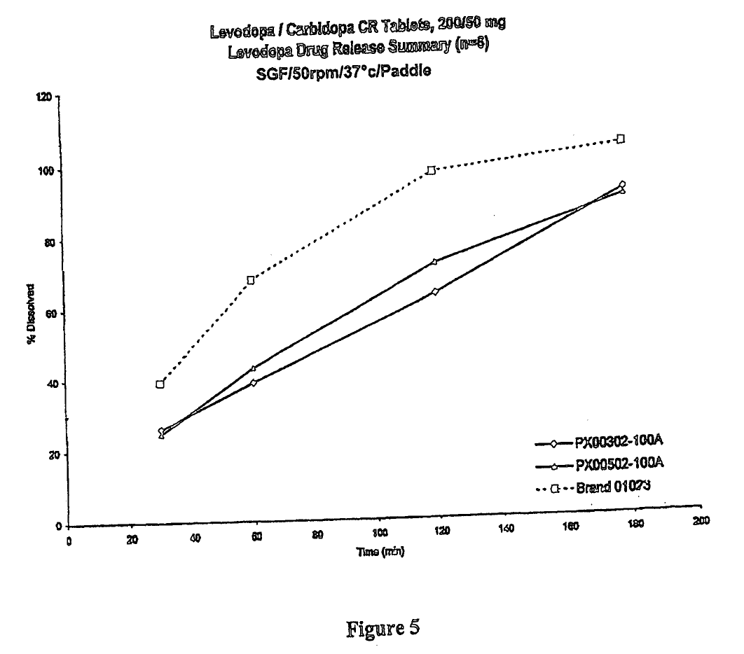

The dissolution sunmaries for carbidopa/levodopa controlled release (CR) 50/200 mg formulations PX00302, PX00502, and SINEMET® CR are shown in Tables 9, 10, and 11, respectively. All data was obtained according to measurements under the USP paddle method of 50 rpm in 900 ml at pH 1.2 (0.1 N HCL) at 37° C. FIG. 5 is a graph of the dissolution of carbidopa/levodopa controlled release (CR) 50/200 mg formulations PX00302, PX00502, and SINEMET® CR.

Quantitative Composition of 50-200 mg CD-LD CR Tablets, Lot PX00302

| | Amount (mg) |
|---|---|
| Components | per tablet |
| Carbidopa, USP | 54 |
| Levodopa, USP | 200 |
| Hydroxypropyl | 19 |
| Cellulose, NF | |
| (Klucel LF) | |
| Lake Blend | 0.7 |
| Purple | |
| (LB-1902) | |
| Magnesium | 5.4 |
| Stearate, NF | |
| Total | 279 |

The PX00302 formulation is prepared in the manner described hereinabove for the PX00502 formulation.

### EXAMPLE 8

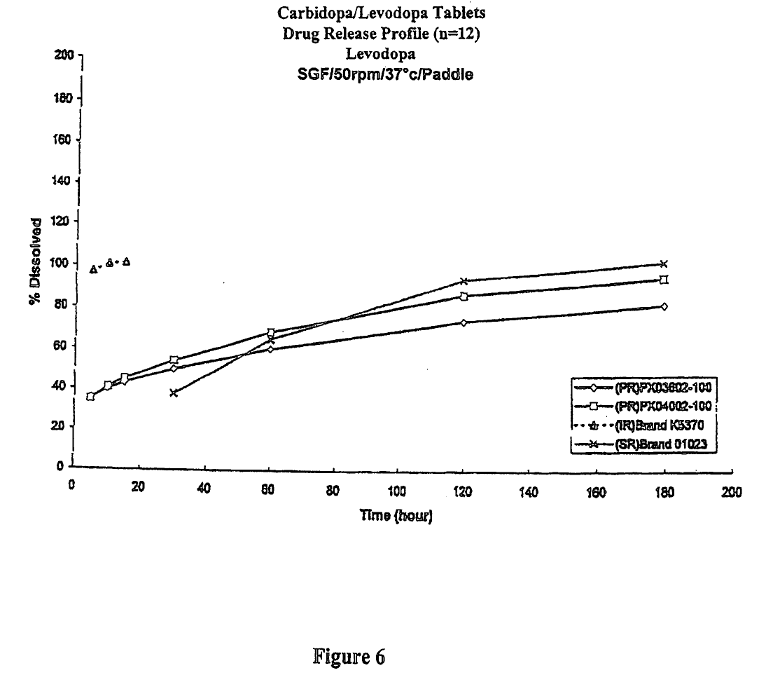

The dissolution summaries for carbidopa/levodopa formulations PX03602 (controlled release, 75/300 mg), PX04002 (controlled release, 75/300 mg), SINEMET® (immediate release, 25/100 mg), and SINEMET® CR (controlled release, 50/200 mg) are shown in Tables 12, 13, 14, and 15, respectively. All data was obtained according to measurements under the USP paddle method of 50 rpm in 900 ml at pH 1.2 (0.1 N HCL) at 37 C. FIG. 6 is a graph of the dissolution profiles of carbidopa/levodopa formulations PX03602 (controlled release, 75/300 mg), PX04002 (controlled release, 75/300 mg), SFNEMET® (immediate release, 25/100 mg), and SINEMET® CR (controlled release, 50/200 mg).

As noted in Example 3, controlled release (or prolonged release (PR)) tablets PX03602 comprise the combination of PX00502(CR) and PX03102, and PR tablets PX04002 comprise the combination of PX00502(CR) and PX03002.

Data For FIG. 4 Levodopa/Carbidopa IR Tablets, 100/25 mg Levodopa Dissolution Summary (n=6) SGF/37° C./50 rpm/Paddle

TABLE 6
Lot PX00102

| | % Dissolved | | | | | | | | | | | | Range | | | |
|---|---|---|---|---|---|---|---|---|---|---|---|---|---|---|---|---|
| Time (min) | V1 | V2 | V3 | V4 | V5 | V6 | V7 | V8 | V9 | V10 | V11 | V12 | Min | Max | Mean | SD |
| 5 | 88 | 82 | 90 | 83 | 89 | 89 | 85 | 87 | 85 | 85 | 68 | 79 | 68 | 90 | 84 | 6.03 |
| 10 | 96 | 90 | 95 | 91 | 95 | 99 | 94 | 96 | 95 | 99 | 89 | 96 | 89 | 99 | 95 | 3.18 |
| 15 | 98 | 92 | 96 | 93 | 97 | 100 | 96 | 97 | 99 | 101 | 91 | 100 | 91 | 101 | 97 | 3.2 |

TABLE 7
Lot PX02001

| | % Dissolved | | | | | | | | | | | | Range | | | |
|---|---|---|---|---|---|---|---|---|---|---|---|---|---|---|---|---|
| Time (min) | V1 | V2 | V3 | V4 | V5 | V6 | V7 | V8 | V9 | V10 | V11 | V12 | Min | Max | Mean | SD |
| 5 | 87 | 96 | 83 | 95 | 80 | 97 | 87 | 89 | 84 | 82 | 90 | 88 | 80 | 97 | 88 | 5.57 |
| 10 | 98 | 98 | 97 | 101 | 92 | 100 | 98 | 98 | 98 | 93 | 98 | 100 | 92 | 101 | 98 | 2.64 |
| 15 | 100 | 99 | 100 | 103 | 97 | 101 | 100 | 100 | 101 | 97 | 100 | 101 | 97 | 103 | 100 | 1.68 |

TABLE 8
Brand (SINEMET ®)

| | % Dissolved | | | | | | | | | | | | Range | | | |
|---|---|---|---|---|---|---|---|---|---|---|---|---|---|---|---|---|
| Time (min) | V1 | V2 | V3 | V4 | V5 | V6 | V7 | V8 | V9 | V10 | V11 | V12 | Min | Max | Mean | SD |
| 5 | 92 | 89 | 97 | 97 | 93 | 97 | 94 | 95 | 92 | 99 | 93 | 102 | 89 | 102 | 95 | 3.62 |
| 10 | 99 | 95 | 100 | 100 | 99 | 102 | 101 | 99 | 99 | 101 | 101 | 104 | 95 | 104 | 100 | 2.05 |
| 15 | 100 | 97 | 101 | 101 | 100 | 103 | 102 | 100 | 101 | 101 | 101 | 104 | 97 | 104 | 101 | 1.69 |

Data for FIG. 5 Levodopa/Carbidopa CR Tablets, 200/50 mg Levodopa Dissolution Summary (n=6) SGF/37° C./50 rpm/Paddle

TABLE 9
PX00302

| | % Dissolved | | | | | | | | | | | | Range | | | |
|---|---|---|---|---|---|---|---|---|---|---|---|---|---|---|---|---|
| Time (min) | V1 | V2 | V3 | V4 | V5 | V6 | V7 | V8 | V9 | V10 | V11 | V12 | Min | Max | Mean | SD |
| 30 | 26 | 26 | 28 | 26 | 25 | 25 | 24 | 26 | 26 | 26 | 26 | 26 | 24 | 28 | 26 | 1.07 |
| 60 | 40 | 39 | 41 | 39 | 37 | 39 | 36 | 38 | 39 | 39 | 39 | 39 | 36 | 41 | 39 | 1.21 |
| 120 | 58 | 62 | 74 | 63 | 56 | 66 | 56 | 57 | 70 | 65 | 58 | 65 | 56 | 74 | 62 | 5.87 |
| 180 | 83 | 90 | 101 | 92 | 75 | 97 | 82 | 87 | 98 | 78 | 93 | 100 | 75 | 101 | 90 | 8.82 |

TABLE 10
PX00502

| | % Dissolved | | | | | | | | | | | | Range | | | |
|---|---|---|---|---|---|---|---|---|---|---|---|---|---|---|---|---|
| Time (min) | V1 | V2 | V3 | V4 | V5 | V6 | V7 | V8 | V9 | V10 | V11 | V12 | Min | Max | Mean | SD |
| 30 | 23 | 24 | 24 | 26 | 25 | 24 | 24 | 25 | 25 | 25 | 24 | 24 | 23 | 26 | 24 | 0.82 |
| 60 | 40 | 43 | 43 | 44 | 45 | 42 | 43 | 44 | 43 | 44 | 42 | 42 | 40 | 45 | 43 | 1.40 |
| 120 | 67 | 71 | 70 | 72 | 75 | 68 | 70 | 73 | 71 | 72 | 69 | 69 | 67 | 75 | 71 | 2.17 |
| 180 | 84 | 88 | 88 | 88 | 91 | 84 | 90 | 93 | 89 | 88 | 86 | 88 | 84 | 93 | 88 | 2.46 |

TABLE 11
SINEMET ® CR

| | % Dissolved | | | | | | | | | | | | Range | | | |
|---|---|---|---|---|---|---|---|---|---|---|---|---|---|---|---|---|
| Time (min) | V1 | V2 | V3 | V4 | V5 | V6 | V7 | V8 | V9 | V10 | V11 | V12 | Min | Max | Mean | SD |
| 30 | 37 | 47 | 42 | 42 | 42 | 34 | 42 | 41 | 30 | 41 | 37 | 34 | 30 | 47 | 39 | 4.81 |
| 60 | 64 | 79 | 71 | 71 | 74 | 59 | 75 | 69 | 53 | 71 | 66 | 60 | 53 | 79 | 68 | 7.69 |
| 120 | 92 | 101 | 99 | 99 | 99 | 93 | 102 | 98 | 84 | 97 | 97 | 93 | 84 | 102 | 96 | 4.86 |
| 180 | 101 | 103 | 103 | 102 | 102 | 105 | 103 | 101 | 103 | 100 | 102 | 104 | 100 | 105 | 102 | 1.55 |

Data for FIG. 6 Levodopa/Carbidopa Compositions Drug Release Summary (n=12), SGF/37° C./50 rpm/Paddle

TABLE 12
(PR, 75/300 mg) PX03602

| | % Dissolved | | | | | | | | | | | | Range | | | |
|---|---|---|---|---|---|---|---|---|---|---|---|---|---|---|---|---|
| Time (min) | V1 | V2 | V3 | V4 | V5 | V6 | V7 | V8 | V9 | V10 | V11 | V12 | Min | Max | Mean | SD |
| 5 | 34.6 | 39.9 | 35.3 | 33.2 | 38.0 | 37.6 | 42.2 | 32.1 | 28.6 | 33.6 | 38.3 | 33.6 | 29 | 42 | 36 | 3.8 |
| 10 | 39.1 | 45.0 | 38.9 | 36.9 | 41.3 | 41.4 | 47.7 | 36.7 | 33.3 | 37.5 | 42.4 | 39.0 | 33 | 48 | 40 | 3.9 |
| 15 | 42.0 | 49.0 | 41.2 | 39.2 | 43.5 | 44.7 | 51.5 | 40.1 | 36.7 | 40.1 | 45.3 | 42.8 | 37 | 52 | 43 | 4.2 |
| 30 | 48.8 | 59.0 | 45.9 | 44.4 | 48.4 | 51.3 | 60.2 | 47.8 | 42.6 | 46.7 | 52.5 | 51.5 | 43 | 60 | 50 | 5.4 |
| 60 | 55.5 | 75.9 | 52.6 | 51.9 | 55.6 | 61.8 | 72.0 | 59.4 | 51.2 | 56.7 | 63.2 | 64.7 | 51 | 76 | 60 | 7.9 |
| 120 | 65.8 | 98.9 | 61.9 | 62.4 | 65.5 | 72.3 | 82.5 | 74.7 | 63.3 | 70.6 | 76.9 | 81.5 | 62 | 99 | 73 | 10.9 |
| 180 | 73.7 | 102.2 | 68.2 | 69.1 | 72.1 | 80.1 | 88.2 | 83.6 | 70.7 | 79.5 | 86.9 | 91.0 | 68 | 102 | 80 | 10.4 |

TABLE 13
(PR, 75/300 mg) PX04002

| | % Dissolved | | | | | | | | | | | | Range | | | |
|---|---|---|---|---|---|---|---|---|---|---|---|---|---|---|---|---|
| Time (min) | V1 | V2 | V3 | V4 | V5 | V6 | V7 | V8 | V9 | V10 | V11 | V12 | Min | Max | Mean | SD |
| 5 | 35.1 | 34.0 | 27.3 | 29.2 | 30.4 | 24.0 | 33.7 | 33.5 | 36.3 | 33.8 | 36.3 | 35.6 | 34 | 36 | 35 | 1.3 |
| 10 | 40.6 | 38.5 | 32.0 | 33.8 | 37.4 | 29.5 | 39.8 | 38.5 | 42.4 | 41.8 | 40.8 | 41.7 | 39 | 42 | 41 | 1.5 |
| 15 | 44.2 | 41.4 | 35.1 | 36.8 | 42.2 | 32.8 | 43.9 | 41.9 | 46.6 | 46.9 | 44.4 | 46.1 | 42 | 47 | 45 | 1.9 |
| 30 | 52.3 | 47.3 | 41.4 | 43.2 | 52.5 | 39.2 | 52.6 | 49.5 | 56.0 | 57.7 | 53.0 | 55.3 | 50 | 58 | 54 | 2.9 |
| 60 | 64.7 | 56.1 | 51.0 | 52.7 | 66.8 | 48.7 | 64.9 | 61.2 | 70.6 | 75.0 | 66.9 | 69.8 | 61 | 75 | 68 | 4.8 |
| 120 | 79.3 | 68.8 | 64.4 | 71.4 | 84.6 | 63.1 | 80.4 | 78.2 | 89.9 | 92.0 | 84.9 | 88.0 | 78 | 92 | 86 | 5.4 |
| 180 | 87.1 | 77.5 | 73.2 | 75.4 | 93.5 | 72.0 | 89.0 | 88.8 | 96.3 | 96.2 | 93.9 | 94.1 | 89 | 96 | 93 | 3.4 |

TABLE 14
(IR 25/100 mg) SINEMET ®

| | % Dissolved | | | | | | | | | | | | Range | | | |
|---|---|---|---|---|---|---|---|---|---|---|---|---|---|---|---|---|
| Time | V1 | V2 | V3 | V4 | V5 | V6 | V7 | V8 | V9 | V10 | V11 | V12 | Min | Max | Mean | SD |
| 5 min | 100.8 | 95.9 | 94.6 | 99.1 | 96.5 | 97.1 | 97.6 | 93.0 | 99.2 | 100.8 | 93.7 | 98.8 | 93 | 101 | 97 | 2.6 |
| 10 min | 101.7 | 99.3 | 100.3 | 102.6 | 100.9 | 100.9 | 99.7 | 98.3 | 101.9 | 103.4 | 98.3 | 100.5 | 98 | 103 | 101 | 1.6 |
| 15 min | 101.6 | 100.7 | 100.7 | 103.1 | 101.8 | 101.3 | 100.7 | 100.7 | 102.0 | 103.6 | 99.6 | 100.7 | 100 | 104 | 101 | 1.1 |

TABLE 15
(SR 50/200 mg) SINEMET ® CR

| | % Dissolved | | | | | | | | | | | | Range | | | |
|---|---|---|---|---|---|---|---|---|---|---|---|---|---|---|---|---|
| Time (min) | V1 | V2 | V3 | V4 | V5 | V6 | V7 | V8 | V9 | V10 | V11 | V12 | Min | Max | Mean | SD |
| 30 | 45.9 | 41.2 | 36.5 | 39.0 | 36.3 | 35.7 | 40.5 | 36.7 | 36.5 | 42.5 | 30.3 | 30.5 | 30 | 46 | 38 | 4.6 |
| 60 | 77.2 | 72.0 | 62.3 | 65.9 | 61.5 | 60.6 | 69.7 | 61.9 | 62.0 | 74.7 | 53.4 | 54.3 | 53 | 77 | 65 | 7.5 |
| 120 | 98.9 | 98.9 | 91.7 | 94.1 | 89.1 | 88.8 | 95.7 | 90.2 | 89.8 | 103.9 | 85.7 | 85.4 | 85 | 104 | 93 | 5.7 |
| 180 | 101.3 | 103.1 | 101.4 | 100.8 | 99.2 | 98.1 | 99.2 | 99.7 | 99.4 | 104.6 | 101.3 | 99.1 | 98 | 105 | 101 | 1.9 |

### EXAMPLE 9

A bilayer tablet was formulated with the ingredients in their respective amounts as shown in Table 16. For the IR layer, carbidopa, levodopa, microcrystalline cellulose, and croscarmellose sodium were granulated in a high-shear granulator using a starch paste. Granules were dried in an oven and screened through US standard mesh screen. Oversized granules were then milled. Screened and milled granules were blended with crospovidone, and then with magnesium stearate. For the ER layer, all ingredients, except magnesium stearate, were granulated in a high-shear granulator with water. Granules were dried in an oven and screened through US standard mesh screen. Oversized granules were then milled. Screened and milled granules were blended with magnesium stearate. The two blends (IR and ER) were then compressed into bi-layer tablets using a rotary tablet press.

TABLE 16
Quantitative Composition of 75-300 mg CD-LD
IR//ER Bilayer Tablet

| | |
|---|---|
| Components | mg per tablet |

ER Layer

| | |
|---|---|
| Carbidopa monohydrate, | 54 |
| USP | |
| Levodopa, USP | 200 |
| Hydroxypropyl Cellulose, | 39 |
| NF | |
| Lake Blend | 1 |
| Magnesium Stearate, NF | 5 |

IR Layer

| | |
|---|---|
| Carbidopa monohydrate, | 27 |
| USP | |
| Levodopa, USP | 100 |
| Microcrystalline Cellulose, | 22 |
| NF | |
| Starch, NF | 4 |
| Croscarmellose Sodium, | 4 |
| NF | |
| Crospovidone, NF | 3 |
| Magnesium Stearate, NF | 2 |

Note that 81 mg carbidopa monohydrate corresponds to 75 mg anhydrous carbidopa.

The same procedure was used to prepare other dosage strengths as shown in the following Table 17.

TABLE 17
Bilayer tablet dosages

| Tablet | Immediate-Release Layer | Extended-Release Layer | Ratio |
|---|---|---|---|
| Strengths | Carbidopa-Levodopa | Carbidopa-Levodopa | IR:ER |
| 100 | 12.5-50 | 12.5-50 | 1:1 |
| 150 | 12.5-50 | 25-100 | 1:2 |
| 200 | 25-100 | 25-100 | 1:1 |
| 250 | 12.5-50 | 50-200 | 1:4 |
| 300 | 25-100 | 50-200 | 1:2 |

### EXAMPLE 10

A bilayer tablet formulation was prepared with the composition shown in Table 18. Similar to the process in the Example 3, for each of the layers, all ingredients, except magnesium stearate, were granulated in a high-shear granulator with water. Granules were dried in an oven and screened through US standard mesh screen. Oversized granules were then milled. Screened and milled granules were blended with magnesium stearate. The blend was compressed into bi-layer tablets using a rotary tablet press.

TABLE 18
Quantitative Composition of 75-300 mg CD-LD IR//ER Bilayer Tablets

| | |
|---|---|
| Components | mg per tablet |

IR Layer

| | |
|---|---|
| Carbidopa monohydrate, | 27 |
| USP | |
| Levodopa, USP | 100 |
| Microcrystalline Cellulose, | 79 |
| NF | |
| Starch, NF | 79 |
| Magnesium Stearate, NF | 4 |

ER Layer

| | |
|---|---|
| Carbidopa monohydrate, | 54 |
| USP | |
| Levodopa, USP | 200 |
| Hydroxypropyl Cellulose, | 39 |
| NF | |
| Lake Blend Purple | 1 |
| Magnesium Stearate, NF | 5 |

### EXAMPLE 11

The process described in Example 10 was used to prepare a bilayer tablet formulation with the composition shown in Table 19.

TABLE 19
Quantitative Composition of 75-300 mg CD-LD IR//ER Bilayer Tablets

| | |
|---|---|
| Components | mg per tablet |

IR Layer

| | |
|---|---|
| Carbidopa monohydrate, | 27 |
| USP | |
| Levodopa, USP | 100 |
| Microcrystalline Cellulose, | 79 |
| NF | |
| Starch, NF | 27 |
| Magnesium Stearate, NF | 4 |

ER Layer

| | |
|---|---|
| Carbidopa monohydrate, | 54 |
| USP | |
| Levodopa, USP | 200 |
| Hydroxypropyl Cellulose, | 39 |
| NF | |
| Lake Blend Purple | 1 |
| Magnesium Stearate, NF | 5 |

### EXAMPLE 12

Bilayer tablet formulations were prepared with the compositions shown in Tables 20 and 21. For the IR layer, carbidopa, levodopa, microcrystalline cellulose, and croscarmellose sodium were granulated in a fluid bed granulator using a starch paste. Granules were screened through US standard mesh screen. Oversized granules were then milled. Screened and milled granules were blended with crospovidone, and then with magnesium stearate. For the ER layer, all ingredients, except magnesium stearate, were granulated in a high-shear granulator with water. Granules were dried in an oven and screened through US standard mesh screen. Oversized granules were then milled. Screened and milled granules were blended with magnesium stearate. The two blends (IR and ER) were then compressed into bi-layer tablets using a rotary tablet press.

TABLE 20
Quantitative Composition of 50-200 mg CD-LD IR//ER Bilayer Tablets
IR Layer

| | |
|---|---|
| Carbidopa monohydrate, | 27 |
| USP | |
| Levodopa, USP | 100 |
| Microcrystalline Cellulose, | 7 |
| NF | |
| Starch, NF | 4 |
| Croscarmellose Sodium, | 4 |
| NF | |
| Crospovidone, NF | 3 |
| Magnesium Stearate, NF | 2 |

ER Layer

| | |
|---|---|
| Carbidopa monohydrate, | 27 |
| USP | |
| Levodopa, USP | 100 |
| Hydroxypropyl Cellulose, | 19 |
| NF | |
| Lake Blend Purple | 0.5 |
| Magnesium Stearate, NF | 3 |

TABLE 21
Quantitative Composition of 50-100 mg CD-LD IR//ER Bilayer Tablets

| | |
|---|---|
| Components | mg per tablet |

IR Layer

| | |
|---|---|
| Carbidopa monohydrate, | 27 |
| USP | |
| Levodopa, USP | 50 |
| Microcrystalline Cellulose, | 40 |
| NF | |
| Starch, NF | 40 |
| Magnesium Stearate, NF | 2 |

ER Layer

| | |
|---|---|
| Carbidopa monohydrate, | 27 |
| USP | |
| Levodopa, USP | 50 |
| Hydroxypropyl Cellulose, | 19 |
| NF | |
| Lactose, NF | 39 |
| Lake Blend Purple | 0.4 |
| Magnesium Stearate, NF | 3 |

### EXAMPLE 11

A bilayer tablet formulation was prepared with the composition shown in Table 22. Similar to the process in the Example 3, for each of the layers, all ingredients except magnesium stearate were granulated in a high-shear granulator with water. Granules were dried in an oven and screened through US standard mesh screen. Oversized granules were then milled. Screened and milled granules were blended with magnesium stearate. The blend was then compressed into bilayer tablets using a rotary tablet press.

TABLE 22
Quantitative Composition of 50-100 mg CD-LD IR//ER Bilayer Tablets

| | |
|---|---|
| Components | mg per tablet |

IR Layer

| | |
|---|---|
| Carbidopa, USP | 27 |
| Levodopa, USP | 50 |
| Microcrystalline Cellulose, | 40 |
| NF | |
| Starch, NF | 40 |
| Magnesium Stearate, NF | 2 |

ER Layer

| | |
|---|---|
| Carbidopa, USP | 27 |
| Levodopa, USP | 50 |
| Hydroxypropyl Cellulose, | 19 |
| NF | |
| Lactose, NF | 4 |
| Lake Blend Purple | 0.4 |
| Magnesium Stearate, NF | 3 |

### EXAMPLE 12

The 75 mg/300 mg (carbidopa/levodopa) bilayer formulation prepared according to Example 9 was administered in randomized, single-dose, two-way crossover study under fasting and fed conditions in 24 healthy volunteers. The study evaluated the effect of a standardized, high-fat, high-calorie meal on the rate and extent of absorption of a single oral dose of the bilayer tablet. Each human subject received one dose in each of two dosing periods (total of two doses per subject), with a washout period of at least seven days between doses. Those subjects randomized into the fed cohort were fed 30 minutes before dosing a standardized high-fat, high-calorie breakfast consisting of: two eggs fried in butter, two slices of toast with butter, eight fluid ounces of whole milk, two strips of bacon and four ounces of hash brown potatoes (standardized FDA high fat meal). Samples were collected from each subject within one hour prior to dosing (0 hour) and after dose administration at: 0.12, 0.25, 0.5, 0.75, 1, 1.5, 2, 2.5, 3, 3.5, 4, 4.5, 5, 6, 7, 8, 10 and 12 hours.

The results are shown in FIG. 7 and Tables 1 and 2. The results show that food has no significant effect on the Cₘₐₓ and AUC of levodopa from a pharmaceutical dosage form according to the invention, (bilayer tablets designated “IPX054“), as seen from the Table 1 (Pharmacokinetic Parameters) and Table 2 (Bioequivalence). The high-calorie, high-fat meal reduces the carbidopa Cₘₐₓ and AUC values of IPX054 by approximately 50%.

### EXAMPLE 13

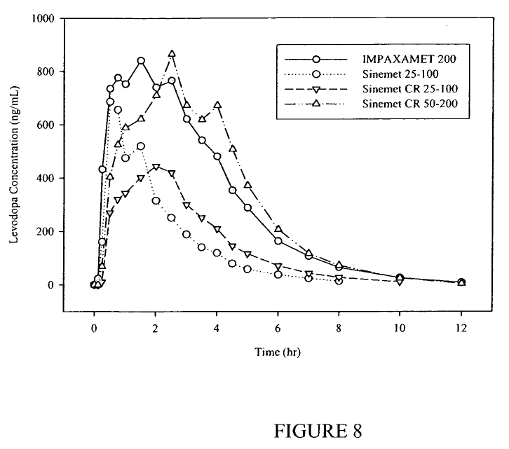

The pharmacokinetic profile of a single oral dose of a 50 mg/200 mg (carbidopa/levodopa) bilayer formulation prepared according to Example 9 was compared to SINEMET® 25-100, SINEMET® CR 25-100 and SINEMET® CR 50-200 by administration to healthy volunteers (range 19-69 years) under fasting conditions. The results are shown in FIG. 8.

### EXAMPLE 14

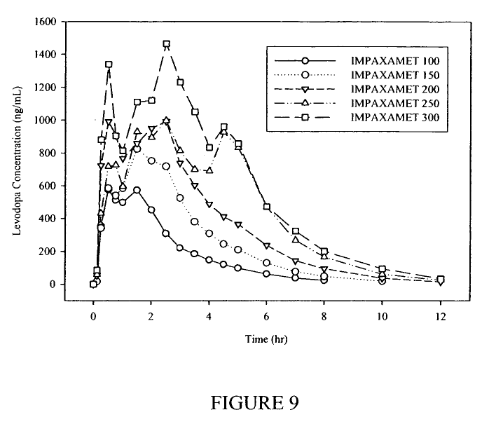

The pharmacokinetic profiles of levodopa after oral dosing of the five dosage strength shown in Example 9 were assessed in healthy subjects 45 to 75 years of age (median: 57 years). The results are shown in FIG. 9 and Table 23 below. The absorption characteristics in these older subjects are similar to those observed in younger healthy subjects, with the time of first plasma levodopa peak (Tₚₑₐₖ₁) occurring at 0.5 hours and Tₘₐₓ occurring at about 0.75 to 2.5 hours. Over the five dosage strengths tested, plasma levodopa Cₘₐₓ and AUC_{0-∞} values increase nearly dose proportionally, and the levodopa Cₚₑₐₖ₁ ranged approximately 2-fold between the highest and lowest tablet strengths.

TABLE 23
Pharmacokinetic parameters of levodopa in healthy subjects (45-75 years)
following a single oral dose under fasting conditions

| IPX054 | Cₘₐₓ | tₘₐₓ | Cₚₑₐₖ₁ | tₚₑₐₖ₁ |
|---|---|---|---|---|
| Strengths | (ng/mL) | (hr) | (ng/mL) | (hr) |
| 100 | 851 ± 280 | 0.75 | 739 ± 338 | 0.5 |
| 150 | 1140 ± 365 | 1.5 | 945 ± 498 | 0.5 |
| 200 | 1522 ± 379 | 1.5 | 1205 ± 518 | 0.5 |
| 250 | 1606 ± 544 | 2.5 | 951 ± 404 | 0.5 |
| 300 | 2142 ± 777 | 1.75 | 1399 ± 506 | 0.5 |

Mean ± SD for Cₘₐₓ and AUC, median for tₘₐₓ and tₚₑₐₖ₁; N = 20

The maximum concentration of carbidopa was reached with a median Tₘₐₓ of 3.5 hours (range: 1-6 hours). The Cₘₐₓ and AUC values of carbidopa increase in a dose of proportional manner from 25 to 75 mg, with the mean Cₘₐₓ values of carbidopa ranging from approximately 125 to 329 ng/mL and the mean AUC_{0-∞} values ranging from approximately 666 to 1846 ng hr/mL across the five strengths.

What is claimed is:

1. A pharmaceutical dosage form having an immediate release component and a controlled release component comprising:
a) an immediate release component comprising a ratio of carbidopa to levodopa of from about 1:1 to about 1:10 such that the in vitro dissolution rate of the immediate release component according to measurements under the USP paddle method of 50 rpm in 900 ml aqueous buffer at pH 4 at 37° C. is from about 10% to about 99% levodopa released after 15 minutes and from about 60% to about 99% after 1 hour; and
b) a controlled release component comprising a ratio of carbidopa to levodopa of from about 1:1 to about 1:10, such that the in vitro dissolution rate of the controlled release component according to measurements under the USP paddle method of 50 rpm in 900 ml aqueous buffer at pH 4 at 37° C. is from about 10% to about 60% levodopa released after 1 hour; from about 25% to about 80% levodopa released after 2 hours; from about 30% to about 85% levodopa released after 4 hours; and from about 40% to about 99% levodopa release after about 6 hours; and
wherein the ratio of levodopa in the immediate release component to levodopa in the controlled release component is from 1:1 to 1:6.

2. The pharmaceutical dosage form according to claim 1, wherein a mean maximum plasma levodopa level of the initial peak (Cₚₑₐₖ₁) from about 400 ng/ml to about 1900 ng/ml occurs after a single oral administration of said dosage form under fasting conditions.

3. The pharmaceutical dosage form according to claim 2, wherein a mean maximum plasma levodopa level of the initial peak (Cₚₑₐₖ₁) from about 700 ng/ml to about 1500 ng/ml occurs after a single oral administration of said dosage form under fasting conditions.

4. The pharmaceutical dosage form according to claim 1, wherein an initial peak plasma level of levodopa obtained in vivo occurs between 0.1 and 0.75 hours after administration of the dosage form to a patient under fasting conditions.

5. The pharmaceutical dosage form according to claim 4, wherein the initial peak plasma level of levodopa obtained in vivo occurs between 0.4 and 0.7 hours after administration of the dosage form to a patient under fasting conditions.

6. The pharmaceutical dosage form according to claim 1, wherein the ratio of levodopa in the immediate release component to levodopa in the controlled release component is between 1:1 and 1:4 inclusive.

7. The pharmaceutical dosage form according to claim 6, wherein the ratio of levodopa in the immediate release component to levodopa in the controlled release component is between 1:1 and 1:2 inclusive.

8. The pharmaceutical dosage form according to claim 1, wherein the immediate release component comprises a ratio of carbidopa to levodopa of from about 1:1 to about 1:3.

9. The pharmaceutical dosage form according to claim 1, wherein the immediate release component comprises a ratio of carbidopa to levodopa of from about 1:4 to about 1:10.

10. The pharmaceutical dosage form according to claim 1, wherein the controlled release component comprises a ratio of carbidopa to levodopa of from about 1:1 to about 1:3.

11. The phanraceutical dosage form according to claim 1, wherein the controlled release component comprises a ratio of carbidopa to levodopa of from about 1:4 to about 1:10.

12. The pharmaceutical dosage form according to claim 1, containing between about 25 mg carbidopa to about 75 mg carbidopa and containing between about 100 mg levodopa and about 300 mg levodopa.

13. The pharmaceutical dosage form according to claim 1, containing between about 10 mg carbidopa to about 25 mg carbidopa and containing between about 50 mg levodopa and about 75 mg levodopa

14. The pharmaceutical dosage form according to claim 1, wherein the dosage form is comprised of particles.

15. The pharmaceutical dosage form according to claim 1, wherein the dosage form is a tablet.

16. The pharmaceutical dosage form according to claim 15, wherein the dosage form is a bilayer tablet.

17. The pharmaceutical dosage form according to claim 1, wherein the carbidopa and levodopa amounts in the immediate release component are 12.5 mg and 50 mg, respectively, and the carbidopa and levodopa amounts in the controlled release component are 12.5 mg and 50 mg, respectively.

18. The pharmaceutical dosage form according to claim 1, wherein the carbidopa and levodopa amounts in the immediate release component are 12.5 mg and 50 mg, respectively, and the carbidopa and levodopa amounts in the controlled release component are 25 mg and 100 mg, respectively.

19. The pharmaceutical dosage form according to claim 2, wherein the carbidopa and levodopa amounts in the immediate release component are 25 mg and 100 mg, respectively, and the carbidopa and levodopa amounts in the controlled release component are 25 mg and 100 mg, respectively.

20. The pharmaceutical dosage form according to claim 1, wherein the carbidopa and levodopa amounts in the immediate release component are 12.5 mg and 50 mg, respectively, and the carbidopa and levodopa amounts in the controlled release component are 50 mg and 200 mg, respectively.

21. The pharmaceutical dosage form according to claim 1, wherein the carbidopa and levodopa amounts in the immediate release component are 25 mg and 100 mg, respectively, and the carbidopa and levodopa amounts in the controlled release component are 50 mg and 200 mg, respectively

22. The pharmaceutical dosage form according to claim 1, wherein the pharmaceutical dosage form has a 90% confidence interval within 0.8 to 1.25 for the fed/fasting ratio of log-transformed AUC and Cₘₐₓ after a single oral administration.

23. A method of reducing intra-patient variability of plasma levodopa levels under fed and fasting conditions during carbidopa/levodopa therapy in a patient suffering from a pathology characterized by reduced levels of dopamine in a patient's brain, said method comprising administering to the patient a pharmaceutical dosage form comprising carbidopa and levodopa in a ratio of from about 1:1 to about 1:10, inclusive, wherein:
the in vitro dissolution rate of the pharmaceutical dosage form according to measurements under the USP paddle method of 50 rpm in 900 ml aqueous buffer at pH 1.2 at 37° C. is from about 20% to about 60% levodopa released after 5 minutes, from about 40% to about 70% released after 30 minutes, and from about 50% to about 80% released after 1 hour.

24. A method according to claim 23, wherein the ratio of carbidopa to levodopa is from about 1:1 to about 1:3, inclusive.

25. A method according to claim 23, wherein the ratio of carbidopa to levodopa is from about 1:4 to about 1:10, inclusive

26. A method according to claim 23, wherein the pharmaceutical dosage form comprises an immediate release component and a controlled release component, wherein each of the immediate release component and controlled release component comprise carbidopa and levodopa in a ratio of about 1:1 to about 1:10, inclusive; and wherein the ratio of levodopa in the immediate release component to levodopa in the controlled release component is from about 1:1 to about 1:6, inclusive.

27. A method according to claim 26, wherein the ratio of levodopa in the immediate release component to levodopa in the controlled release component is between 1:1 and 1:4 inclusive.

28. A method according to claim 26, wherein the controlled release component comprises a matrix material for controlling the release of the carbidopa and levodopa from said controlled release component, said matrix material comprising a gel-forming polymer.

29. A method according to claim 28, wherein the gel-forming polymer is a cellulose ether.

30. A method according to claim 29, wherein the cellulose ether is hydroxypropyl cellulose.

31. A method according to claim 28, wherein the dosage form is a tablet.

32. A pharmaceutical dosage form having an immediate release component and a controlled release component:
a) wherein both the immediate release component and the controlled release component comprise carbidopa and levodopa;
b) wherein the ratio of carbidopa to levodopa in the pharmaceutical dosage from is from about 1:1 to about 1:10 inclusive;
c) wherein the pharmaceutical dosage form comprises at least about 200 mg levodopa; and
d) wherein upon oral administration of the pharmaceutical dosage form, the plasma level of levodopa is at least about 600 ng/ml within 30 minutes after administration, and said levodopa plasma level is maintained above 600 ng/ml for at least about 3.5 hours.

33. A pharmaceutical dosage form according to claim 32, wherein the ratio of carbidopa to levodopa is from about 1:1 to about 1:3 inclusive.

34. A pharmaceutical dosage form according to claim 32, wherein the ratio of carbidopa to levodopa is from about 1:4 to about 1:10 inclusive.

35. A pharmaceutical dosage form having an immediate release component and a controlled release component:
a) wherein both the immediate release component and the controlled release component comprise carbidopa and levodopa;
b) wherein the controlled release component comprises a matrix material for controlling the release of the carbidopa and levodopa from said controlled release component; and
c) wherein the matrix material comprises a gel-forming polymer in an amount such that the in vitro dissolution rate of the controlled release component according to measurements under the USP paddle method of 50 rpm in 900 ml aqueous buffer at pH 4 at 37° C. is from about 10% to about 60% levodopa released after 1 hour; from about 25% to about 80% levodopa released after 2 hours; from about 30% to about 85% levodopa released after 4 hours; and from about 40% to about 99% levodopa release after about 6 hours.

36. A pharmaceutical dosage form according to claim 35, wherein the gel-forming polymer is a cellulose ether.

37. A pharmaceutical dosage form according to claim 36, wherein the cellulose ether is hydroxypropyl cellulose.

38. A pharmaceutical dosage form according to claim 35, wherein the dosage form is a tablet.

39. A pharmaceutical dosage form according to claim 38, wherein the controlled-release component is present in a tablet core and the immediate-release component is present as a coating over said controlled-release core.
